(19) United States
(12) Patent Application Publication
(10) Pub. No.: US 2026/0096897 A1
(43) Pub. Date: Apr. 9, 2026

(54) METHODS TO RECONSTRUCT BONE AND JOINT SURFACES IN A SHOULDER JOINT

(71) Applicant: SUBCHONDRAL SOLUTIONS, INC., Los Gatos, CA (US)

(72) Inventors: Derek Dee, Rancho Palos Verdes, CA (US); Chris Maurer, Solana Beach, CA (US)

(73) Assignee: SUBCHONDRAL SOLUTIONS, INC., Los Gatos, CA (US)

(21) Appl. No.: 19/113,863

(22) PCT Filed: Sep. 21, 2023
(86) PCT No.: PCT/US2023/074798

§ 371 Date: Mar. 20, 2025

Related U.S. Application Data
(60) Provisional application No. 63/408,771, filed on Sep. 21, 2022.

(51) Int. Cl. A61F 2/40; A61F 2/30; A61F 2/46

(52) U.S. Cl. CPC A61F 2/4081; A61F 2/30749; A61F 2/4014; A61F 2/4601; A61F 2/4612; A61F 2002/30224; A61F 2002/30331; A61F 2002/30461; A61F 2002/30514; A61F 2002/30622; A61F 2002/30677; A61F 2002/4677

(57) ABSTRACT

Bone and joint surface reconstruction methods are disclosed for the therapeutic amelioration of joint defects caused by various conditions, injuries, and diseases to help eliminate or reduce pain and return the joint to its proper bio-mechanical function without the need to replace fully or partially the anatomical joint. The joint may specifically include mammalian joints such as the glenohumeral joint or other joint of the shoulder, for example. The methods disclosed herein leverage the significant role the subchondral bone plays in the health status of the afflicted joint.

### BACKGROUND

We are at a point in history where people are living longer, more active lives. Boys, girls, men, and women are exercising and playing sports year-round. However, this prolonged activity can take a toll on human body joints, such as joints in the shoulder. Repeated stress to a damaged joint caused by injury or just normal activity can lead to pain and swelling in the joint. This stress can cause damage including fractures to the bone surrounding the injury. The damage to the bone is largely responsible for the pain.

Non-surgical joint treatments usually involve pharmacological intervention such as the administration of non-steroidal anti-inflammatory drugs or injection of hyaluronic acid-based products. These treatments are initially administered to patients experiencing relatively less severe pain or joint complications. However, when non-surgical treatments prove ineffective, or for patients with severe pain or bone injury, invasive surgical intervention is most likely required.

However, none of these treatments ameliorate all the joint defects (i.e., conditions, injuries and diseases). There is accordingly a need for more efficient ways to treat these areas where cartilage is reduced or absent. Therefore, there is a legitimate need for less invasive methods of reconstructing bone and joint surfaces while preserving a joint in the shoulder with an implant that also encourages tissue regrowth at the surgical site.

### BRIEF SUMMARY

One general aspect of the present disclosure includes a surgical assembly configured for use in a surgery to be performed relative to a glenohumeral or other joint of the shoulder for intervening in subchondral bone therein to ameliorate one or more defects. The surgical assembly also includes a driver instrument. The assembly also includes a hybrid subchondral implant-biologic member construct configured to be installed relative to the glenohumeral or other joint of the shoulder and may include: a biologic member having a slit formed therethrough, where the slit is arranged so that the driver is extending through the slit, where the biologic member is tailored to provide therapeutic effect in the glenohumeral or other joint of the shoulder; and a subchondral implant engaged with the driver, where the subchondral implant is sized and shaped to be received into the subchondral bone of the glenohumeral or other joint of the shoulder, where the biologic member is secured to the subchondral implant so that the hybrid subchondral implant-biologic member construct is formed on the driver to facilitate installation into the glenohumeral or other joint of the shoulder by operation of the driver.

Implementations may include one or more of the following features. The surgical assembly where the subchondral implant and the biologic member are configured to be installed as the hybrid subchondral implant-biologic member construct over a top of a central post formed by removing a circular portion of the subchondral bone in the glenohumeral or other joint of the shoulder while preserving the central post of the subchondral bone in a substantially undisturbed native state. The subchondral implant may include a cylindrical body having a leading portion and a trailing portion, where the leading portion defines an open bottom sized for receiving the central post, and where the trailing portion may include an inset wall inset toward a longitudinal axis of the body and away from an outermost perimeter of the body, where the inset wall is sized and arranged for receiving the biologic member in an arrangement in which the biologic member is folded such that a first part of the biologic member is received along the inset wall and a second part of the biologic member is received along a top of the trailing portion. The subchondral implant further may include at least one prong extending from the inset wall and sized and configured for engaging the biologic member. The biologic member is retained folded up over a trailing portion of the subchondral implant by at least one suture or other retainer. The surgical assembly may include at least one retainer securing the biologic member such that at least one portion of the biologic member is atop the subchondral implant and at least one other portion of the biologic member is along a lateral side of the subchondral implant. The subchondral implant may include a cylindrical body having a body outer diameter sized and configured to engage portions of multiple bones along a joint for fusing of the joint, where the subchondral implant further may include a washer flange laterally extending from the cylindrical body away from the body outer diameter and to a washer outer diameter sized to engage the bones along the joint for transferring load thereto. The washer flange is formed in a washer coupled with the cylindrical body. The washer flange includes at least one spike arranged to extend into bone and having angle, bevel, and/or other shape that encourages compression of the bone against the implant and other bone to promote healing and fusion. The surgical assembly may include at least one anchor in engagement with a fenestration through the subchondral implant, the anchor coupled with a tether sized and arranged for coupling with the biologic member and/or for coupling with surrounding tissue at an installation site. The surgical assembly may include: a guide wire alignable with the driver instrument; and an alignment attachment may include a base defining a lumen sized for engaging the guide wire, the alignment attachment may include an arm extending from the base and may include an alignment aperture positioned for receiving a secondary tool therethrough so as to align the secondary tool for operation in parallel or adjacent to operation along the guide wire. The hybrid subchondral implant-biologic construct includes a biologic graft of the same size, larger than, or asymmetric with respect to the subchondral implant. At least one of: the biologic graft is attachable to a top and/or side(s) of the subchondral implant; or the biologic graft includes sides and edges, the edges configured to be tucked down to cover one or more sides of the subchondral implant. The method may include placing one or more than one additional hybrid subchondral implant-biologic construct into the one or more defects.

One general aspect of the present application includes a method of intervening in subchondral bone to ameliorate one or more defects while preserving a glenohumeral or other joint of the shoulder using a hybrid subchondral implant-biologic member construct. The method also includes surgically opening access to a glenohumeral or other joint of the shoulder joint of the patient; preparing a site of subchondral bone at the glenohumeral or other joint of the shoulder by removing a circular portion of the subchondral bone while preserving a central post of the subchondral bone in a substantially undisturbed native state, preparing a hybrid subchondral implant-biologic member construct by: forming a slit through a biologic member, positioning a driver instrument through the slit, engaging the driver instrument with a subchondral implant, and securing the biologic member to the subchondral implant so that the hybrid subchondral implant-biologic member construct is formed. The method also includes installing the hybrid subchondral implant-biologic member construct over a top of the central post in the glenohumeral or other joint of the shoulder.

Implementations may include one or more of the following features. The method where the installing may include the hybrid subchondral implant biologic construct may include being introduced in a direction that is an antegrade approach relative to the joint. The installing may include the hybrid subchondral implant biologic construct may include being introduced in a direction that is a retrograde approach relative to the joint. The installing may include the hybrid subchondral implant biologic construct may include being introduced in a direction that is at an angle within 20-90 degrees with respect to a longitudinal axis of the joint or that is at another tangential approach relative to the joint.

One general aspect of the present application includes a method of intervening in subchondral bone to fuse a glenohumeral or other joint of the shoulder using a subchondral implant. The method also includes surgically opening access to a glenohumeral or other joint of the shoulder joint of the patient; preparing a site of subchondral bone at the glenohumeral or other joint of the shoulder by removing damaged tissue in and/or between the subchondral bone of multiple bones along the joint so as to form a bore between the multiple bones along the joint; preparing a subchondral implant; and installing the subchondral implant construct in the bore so that the subchondral implant is engaging the subchondral bone of the multiple bones along the glenohumeral or other joint of the shoulder, where the subchondral implant engaging the subchondral bone of the multiple bones resists relative motion therebetween and thus facilitates fusion of the joint.

Implementations may include one or more of the following features. The method where the subchondral implant may include a cylindrical body having a body outer diameter and a body inner diameter, where the subchondral implant further may include a washer flange laterally extending from the cylindrical body away from the body outer diameter and to a washer outer diameter, where the washer engages surfaces of the multiple bones outward from the bore.

### DRAWINGS

These and other features, aspects and advantages of the present invention will become better understood with regard to the following description, appended claims, and accompanying figures where:

FIG. 1A is a schematic depiction showing elements for intervention in a shoulder joint;

FIG. 1B is a cross sectional histological illustration of a normal healthy joint, an unhealthy joint with early-stage osteoarthritis, and an unhealthy joint with late-stage osteoarthritis;

FIG. 2A is a flow chart illustrating a generalized method that may be utilized as a surgical procedure;

FIG. 2B is a pre-surgical perspective view of a shoulder of a patient according to some examples;

FIGS. 3 and 4 are top views showing open surgical access to a shoulder joint of FIG. 2;

FIG. 5A is a perspective view of a hypertrophic bone with degenerative erosive articular joint disease;

FIG. 5B is a perspective view of a guide pin being advanced toward a defect;

FIG. 6A is a perspective view of a cannulated countersink being advanced along a guide pin toward a defect according to embodiments of methods to reconstruct bone and joint surfaces according to some examples;

FIG. 6B is a perspective view of a cannulated countersink in contact with a defect according to embodiments of methods to reconstruct bone and joint surfaces according to some examples;

FIG. 6C is a perspective view of a guide pin and cannulated countersink being withdrawn from a defect along a guide pin according to embodiments of methods to reconstruct bone and joint surfaces according to some examples;

FIG. 7 is a perspective view measuring cartilage depth post debridement of diseased articular cartilage;

FIG. 8A is a perspective view of a cannulated coring reamer being advanced along a guide pin toward a defect according to embodiments of methods to reconstruct bone and joint surfaces according to some examples;

FIG. 8B is a perspective view of a cannulated coring reamer in contact with a defect according to embodiments of methods to reconstruct bone and joint surfaces according to some examples;

FIG. 8C is a perspective view after a cannulated coring reamer has removed a circular portion of the subchondral bone at a defect while preserving a central post of subchondral bone in a substantially undisturbed native state by simultaneously rotating and lowering the cannulated coring reamer into a center of the countersink hole according some examples;

FIG. 9 is a perspective view of a hybrid subchondral implant-biologic construct being advanced over the guide pin with a driver instrument according to some examples;

FIG. 10A is a radiographic image showing alignment of a shoulder joint and the implanted hybrid subchondral implant-biologic construct in a vertical plane (e.g., a coronal or transverse plane) with the articular defect repaired and the subchondral insufficiency microfractures stabilized according to embodiments of methods to reconstruct bone and joint surfaces according to some examples;

FIG. 10B is a radiographic image showing alignment of the shoulder joint and the implanted hybrid subchondral implant-biologic construct in a horizontal plane (e.g., a sagittal plane) according to embodiments of methods to reconstruct bone and joint surfaces according to some examples;

FIG. 11 is a perspective view of subchondral bone prepared to receive a hybrid subchondral implant-biologic construct in a fully implanted state to stabilize or secure an insufficiency fracture according to some examples;

FIG. 12 is a perspective view of a hybrid subchondral implant-biologic construct implanted with a graft added according to some examples;

FIGS. 13A-13F are cross-sectional diagrams depicting various orientations and methods to fasten a subchondral implant and biologic construct together according to some examples;

FIGS. 14A-14Q are cross-sectional diagrams depicting various approaches and orientations to implant a hybrid subchondral implant-biologic construct into a bone according to embodiments of methods to reconstruct bone and joint surfaces according to some examples;

FIGS. 15A-15L are schematic depictions illustrating operations that may be utilized to prepare the hybrid subchondral implant-biologic construct relative to a driver instrument according to some examples;

FIGS. 16A-16D are schematic depictions illustrating operations that may be utilized to fuse bones using the subchondral implant according to some examples;

FIGS. 17A-17B are schematic depictions illustrating operations that may be utilized for tangential insertion of the subchondral implant along an edge of the joint according to some examples;

FIG. 18 is a schematic depiction illustrating operations that may be utilized for installation to align a portion of the hybrid subchondral implant-biologic construct along an edge of the joint according to some examples;

FIG. 19 is a schematic depiction illustrating anchors that may be utilized for securing relative to the hybrid subchondral implant-biologic construct according to some examples; and

FIG. 20 is a schematic depiction illustrating an alignment attachment that may be utilized according to some examples.

### DESCRIPTION

According to some examples, there is provided a method of reconstructing bone and joint surfaces to ameliorate joint conditions and diseases while preserving a shoulder joint. The methods may utilize a hybrid construct that may include a subchondral implant coupled with a biologic member and which may accordingly be alternatively referred to as a hybrid subchondral implant-biologic construct, a hybrid subchondral implant-biologic member construct, a hybrid construct, or other variations thereof. However, in some scenarios or embodiments, a construct may be utilized that includes a subchondral implant without a biologic member pre-attached (e.g., the biologic member may be omitted or may be added after installation of the subchondral implant). These methods address fractures to the bone and reconstructs the shoulder joint to help eliminate or reduce pain and return the shoulder joint to its proper bio-mechanical function.

In some examples, the method comprises providing a device. The disclosures of U.S. Pat. Nos. 11,298,235; 11,039,927; 10,610,364; 9,532,878; 9,155,625; 8,968,404; and 8,753,401, all assigned to Subchondral Solutions, Inc., are incorporated by reference in their entirety for all purposes. Examples of the device and methods will now be disclosed in detail.

As used in this disclosure, except where the context requires otherwise, the term “comprise” and variations of the term, such as “comprising,” “comprises” and “comprised” are not intended to exclude other additives, components, integers, or steps.

As used in this disclosure, except where the context requires otherwise, the method steps disclosed and shown are not intended to be limiting nor are they intended to indicate that each step is essential to the method or that each step must occur in the order disclosed but instead are exemplary steps only, All dimensions specified in this disclosure are by way of example only and are not intended to be limiting, except where the context requires otherwise. Further, the proportions shown in these Figures are not necessarily to scale. As will be understood by those with skill in the art with reference to this disclosure, the actual dimensions and proportions of any device or part of a device disclosed in this disclosure will be determined by its intended use.

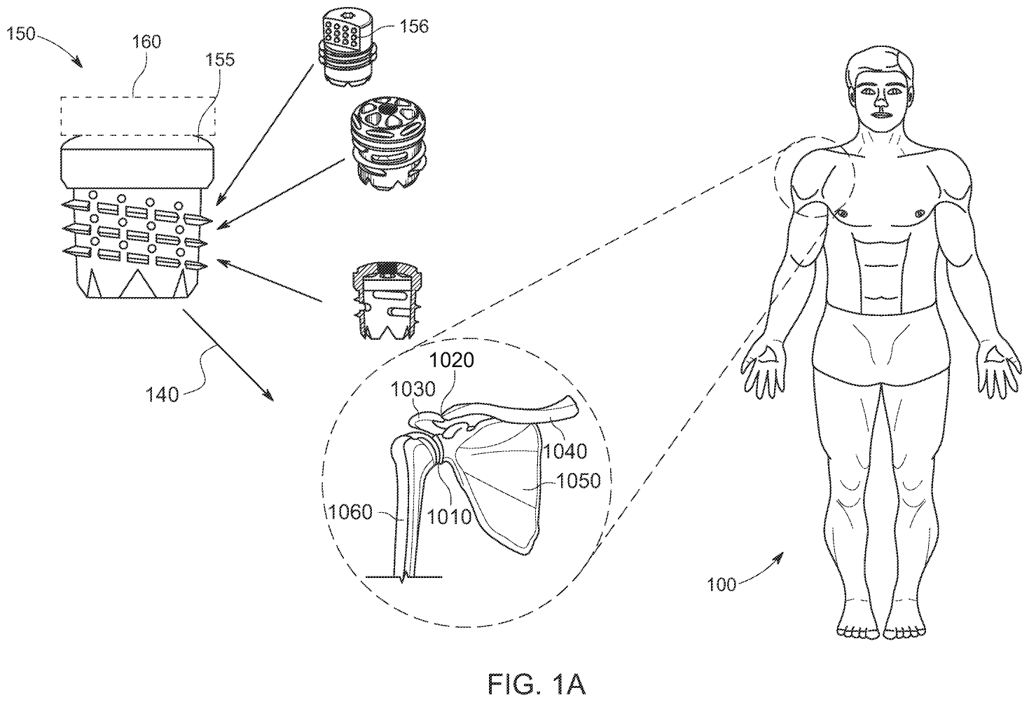

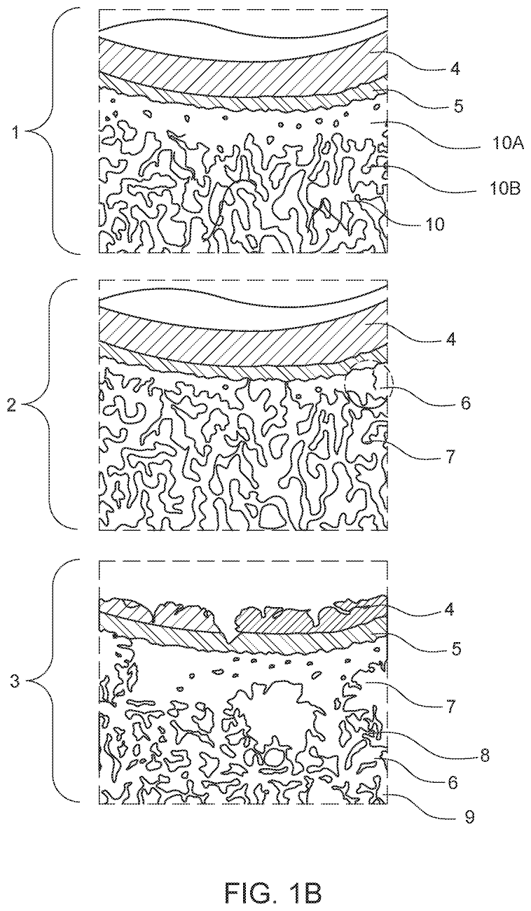

According to some examples, there is provided a method of reconstructing bone and joint surfaces to ameliorate joint conditions and diseases. Referring now to FIG. 1A, the method can be performed relative to shoulder joints, such as the glenohumeral joint 1010 or the acromioclavicular (AC) joint 1020. The glenohumeral joint 1010 can be a joint between the humerus 1060 and the scapula (shoulder blade) 1050. The AC joint 1020 can be a joint between the clavicle 1040 and the acromion 1030. In addition to the glenohumeral joint 1010 and the AC joint 1020, other examples of joints in the shoulder can include the sternoclavicular (SC) and scapulothoracic joints. The joints in the shoulder can include any part of the shoulder where two or more bones meet to allow movement. Bones involved in joints in the can include the acromion 1030, the clavicle 1040, the scapula 1050, the humerus 1060, the sternum, and ribs of the thoracic rib cage. As an example, the SC joint can be a link between the clavicle 1040 and the sternum.

For purposes of the following narrative, examples may be given relative to particular structures of a joint associated with the shoulder; however, the method may be implemented relative to any suitable combination of structures of any joint associated with the shoulder. The method may include installation (e.g., generally denoted by arrow 140) of a hybrid construct 150 into any joint of the shoulder. For example, the hybrid construct 150 can include a subchondral implant 155, which can be coupled with a biologic member 160, such as described in greater detail elsewhere herein.

Various versions of the subchondral implant 155 may be suitable and may differ from one another in combinations and/or configurations of features included. Some varying versions are shown by way of example in FIG. 1A in a column to the right of the generalized depiction of the implant 155 depicted at left in FIG. 1A, although other combinations may be utilized additionally or alternatively. Generally, the subchondral implant 155 can include a body. The body may at least partially approximate a cylindrical shape, such as with an open bottom opposite a top (e.g., which may be at least partially enclosed). Examples of the top and the open bottom may be appreciated with reference to the lowest view in the rightward column of views, for example.

Fenestrations may be included. Fenestrations may include pin-holes, slots, or other shapes. Some examples of such variety is schematically represented in the circular fenestrations in the view at left in FIG. 1A, and with more specific shapes shown by way of example in the rightward column of views in FIG. 1A. The fenestrations may be arranged extending through peripheral walls (e.g., extending radially) and/or through the top (e.g., extending longitudinally).

The body may include an internal cavity, such as may be accessible through the open bottom and/or through the fenestrations. One example of an internal cavity is shown in the cross-sectional view at bottom of the rightward column of views in FIG. 1A.

Threads may be included, such as arranged on an exterior of the body. Various examples of threads are shown in all the depicted views in FIG. 1A, although in some examples, threads may be at least partially or wholly omitted. Some fenestrations may be positioned between threads (such as in the middle right view) and/or as interruptions to thread paths (such as in the left view).

A driver interface may be included (e.g., on the top) and may facilitate engagement with a suitable tool for driving the implant into position during installation. Arrangements of features may correspond to those described in the above-noted disclosures assigned to Subchondral Solutions and/or may correspond to arrangements in which depicted features are omitted or arranged differently. Threads may be omitted or removed to provide smooth engagement surfaces along at least some longitudinally extending portions of the walls in some examples, with one example being shown in the illustrative example at top of the rightward column in FIG. 1A.

The body of the implant 155 may be a cylindrical body. The body may have a leading portion and a trailing portion. For example, in the upper right view, the leading portion may correspond to the portion shown at the bottom of the view, while the trailing portion may be a portion at the top of the view. The leading portion may define an open bottom (e.g., sized for receiving a central post as discussed elsewhere herein). The trailing portion may include a driver interface (e.g., sized for engagement with a driving instrument as discussed elsewhere herein).

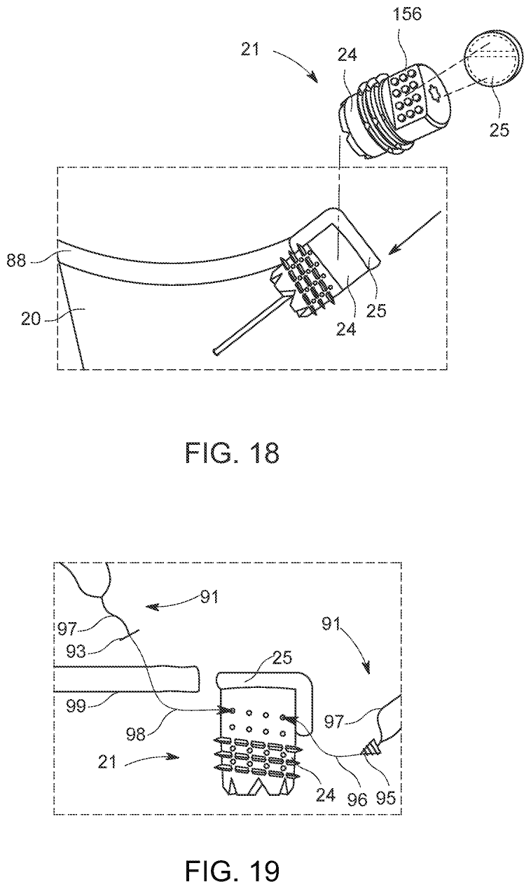

The trailing portion can include an inset wall 156. The inset wall 156 may be inset toward a longitudinal axis of the body and away from an outermost perimeter of the body, for example. The inset wall 156 may be sized and arranged for receiving the biologic member 160, at least in part. For example, the inset wall 156 may receive the biologic member 160 in an arrangement in which the biologic member 160 is folded such that a first part of the biologic member is received along the inset wall 160 and a second part of the biologic member 160 is received along a top of the trailing portion. One such example may be appreciated with further reference to FIG. 18, for example. The inset wall 156 may be aligned along a chord, diameter, or other segment extending along at least a portion of a circle defined by the outermost periphery of the body of the implant 155. The inset wall 156 can be a planar surface or include some curvature or exhibit other contours. The inset wall 156 is further shown with several prongs extending from the inset wall 156 (e.g., which may be sized and configured for engaging the biologic member), although any number may be utilized, including zero (e.g., omitting) or one or more.

Although various features have been described, any combination of more, fewer, and/or different features may be implemented with the implant 155. Additionally or alternatively, the implant 155 may be utilized with any features that may facilitate operations of a method described herein.

By way of example, the method can be performed relative to the glenohumeral joint 1010. The glenohumeral joint 1010 may include the humerus 1060 of the arm and the scapula 1050 of the shoulder. Ligaments associated with the glenohumeral joint 1010 can include the coracohumeral, glenohumeral, and transverse humeral ligaments. Tendons of in a vicinity of the glenohumeral joint 1010 can include the supraspinatus, subscapularis, infraspinus, and teres minor. The glenohumeral joint may be included in the ball and socket class of joints. For purposes of the following narrative, the glenohumeral joint will be referred to as a shoulder joint; however, the following description could be used for any joint associated with the shoulder, including but not limited to the AC, SC, and scapulothoracic joints. The method may include installation (e.g., generally denoted by arrow 140) of a hybrid construct 150 into a shoulder joint. For example, the hybrid construct 150 can include a subchondral implant 155, which can be coupled with a biologic member 160, such as described in greater detail elsewhere herein.

FIG. 1B is a cross sectional histological illustration of a normal joint 1, a joint with early-stage osteoarthritis 2, and a joint with late-stage osteoarthritis 3. The depicted joints 1, 2, and 3 may each correspond to the shoulder joint, for example. FIG. 1B shows the progression of microdamage of articular cartilage and subchondral bone in osteoarthritis. Specifically, the non-calcified cartilage 4 degenerates over time, calcified cartilage 5 thickens, subchondral microdamage 6 occurs, and the subchondral plate loses thickness and increases porosity 7. Often, there is also subchondral trabeculae deterioration in early stage 1 osteoarthritis and subchondral trabeculae sclerosis 9 in late-stage osteoarthritis 3. This often leads to subchondral bone cysts 8.

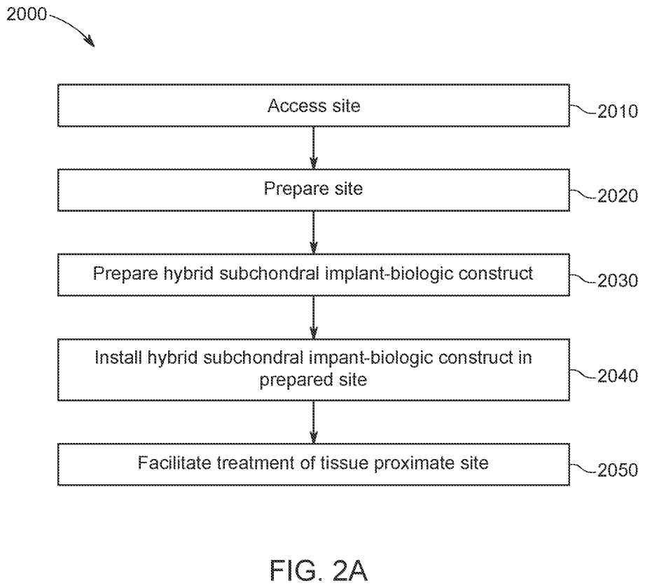

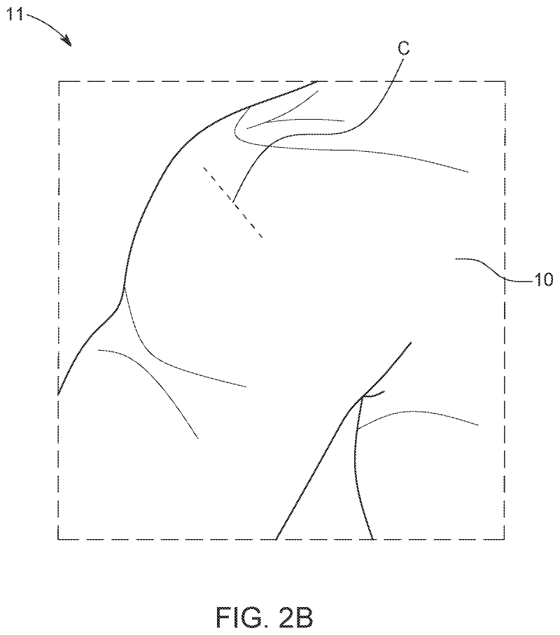

FIG. 2A is a flow chart illustrating a generalized method 2000 that may be utilized as a surgical procedure for a shoulder joint. The method 2000 is described generally initially, and particular examples of application of the method are described afterward.

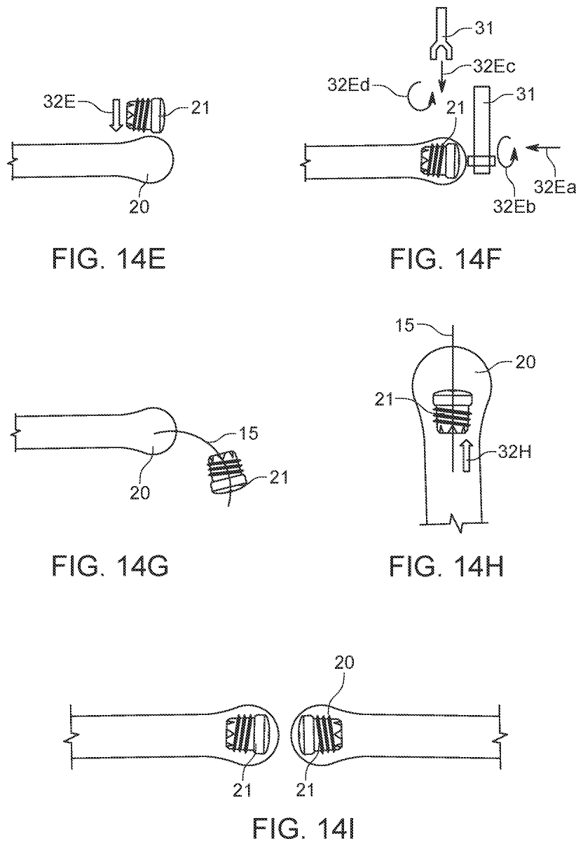

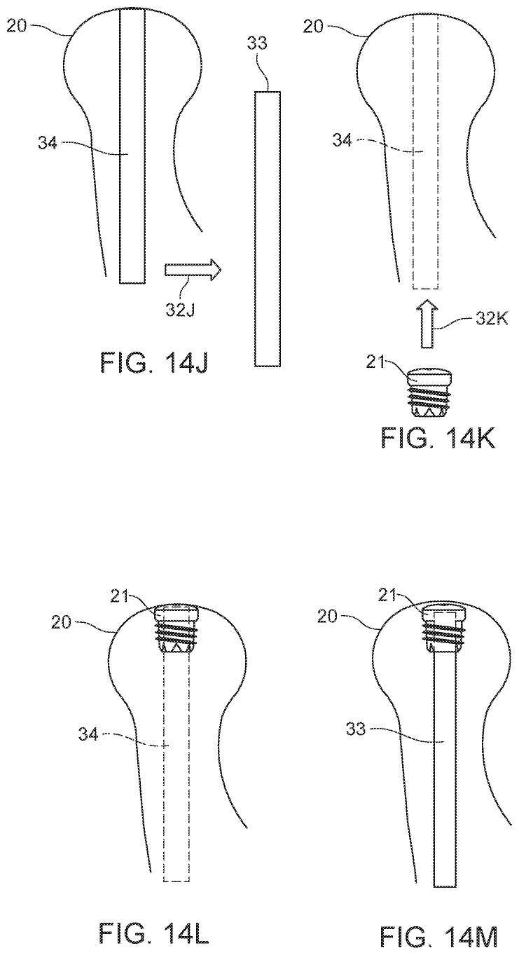

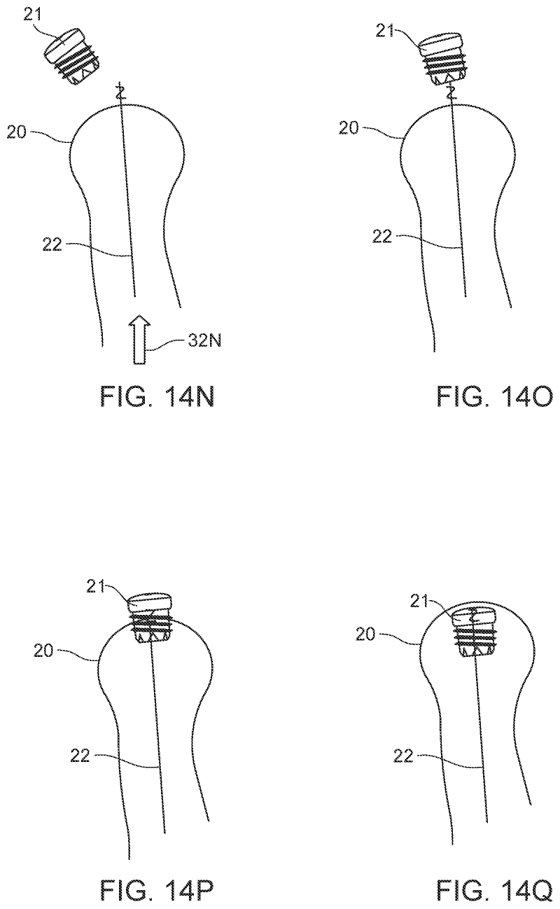

The method 2000 at 2010 can include accessing a surgery site. For example, this may involve accessing a joint of the shoulder, such as the AC joint or sternoclavicular joint. Access may include a minimally invasive or arthroscopic procedure. Access may be characterized and/or affected by factors such as anatomy accessed and/or approach trajectory. The anatomy accessed may correspond to anatomy at the AC joint, for example. Approach trajectory may be specific to the AC joint in some examples. Approach trajectories may correspond to examples discussed with respect to FIGS. 14A-14I, for example. As some non-limiting examples, in a case of repair involving a shoulder joint, an anterior approach may be made, a retrograde approach may be made, or a tangential approach may be made to the scapula, the clavicle, the acromion, the sternum, or the humerus, or to a region between the bones, although approaches additionally or alternatively may include antegrade, retrograde, or tangential through, past, or into one or more of the structures noted in FIG. 1A. As one example, in a case of repair involving the AC joint, an approach may be made past the superior or inferior acromioclavicular ligaments and into the clavicle and/or the acromion. In another example, for a glenoid tangential insertion and approach, an implant graft can be passed from an anterior approach to the shoulder, above the subscapularis tendon. The implant graft can enter the joint past the anteroinferior capsule while retracting the subscapularis tendon.

The method 2000 at 2020 can include preparing the site. For example, this may involve preparing the AC joint. Preparing the site may be characterized and/or affected by factors such as anatomy accessed and/or actions performed for preparing the site. Actions may include acts such as described with respect to FIGS. 2B-8C or comparable or other acts for the respective anatomy accessed, for example. As one example, in a case of repair involving the AC joint, preparing may include temporarily displacing and splitting the deltoid muscle and fascia and the superior capsular ligaments for exposing a suitable path to the clavicle, the acromion, both, or a region between both, for a superior approach, or alternatively, splitting the deltoid from a lateral subacromial approach, releasing the inferior AC capsular ligaments, and subluxing the clavicle inferiorly so as to approach through the subacromial space

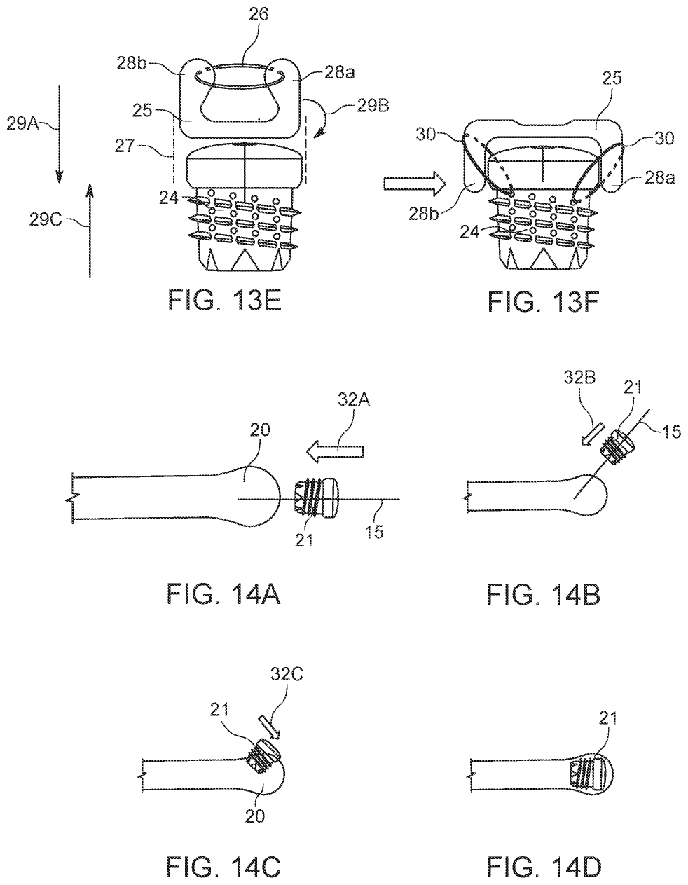

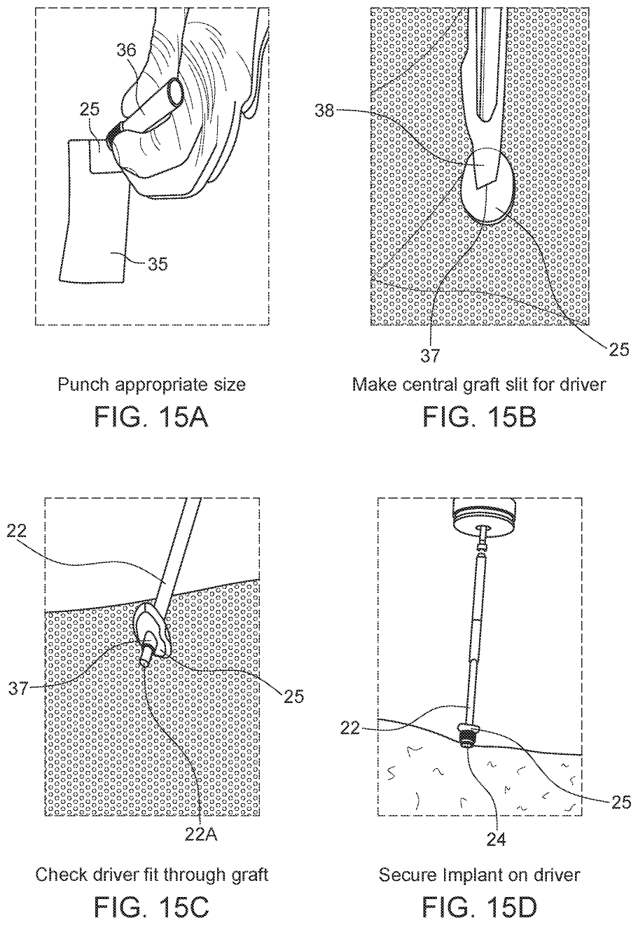

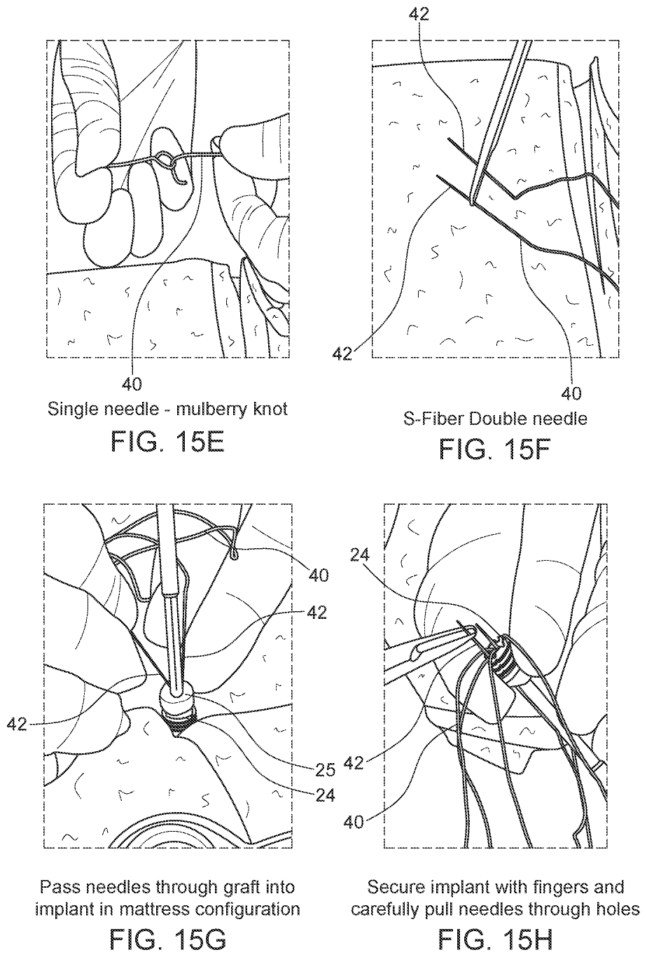

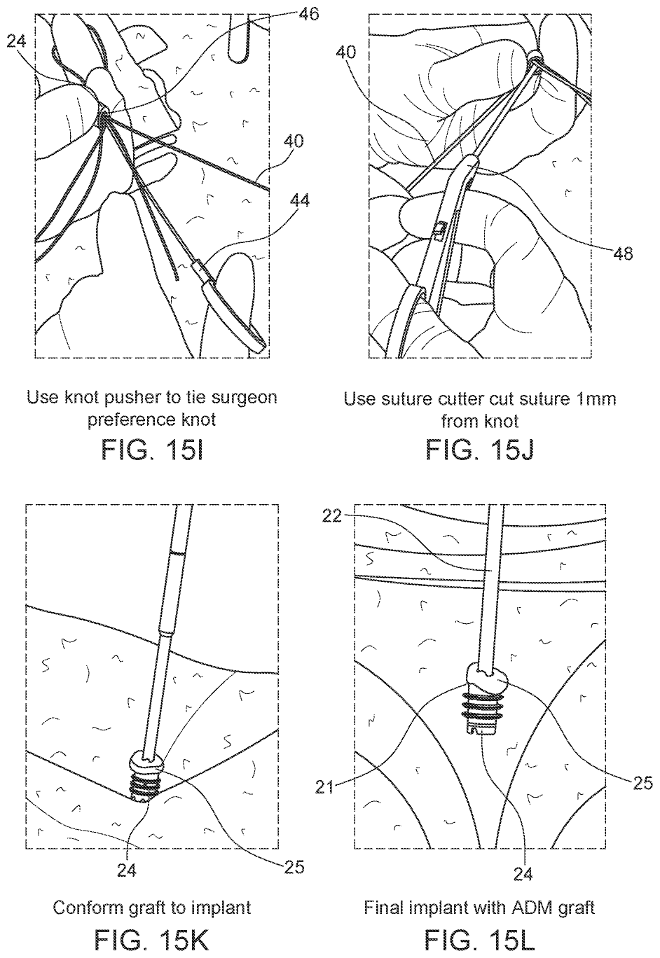

The method 2000 at 2030 can include preparing a hybrid subchondral implant-biologic construct. This can correspond to acts performed relative to a hybrid subchondral implant-biologic construct 21 described with respect to FIGS. 13A-13F and/or FIGS. 15A-L, for example. The hybrid subchondral implant-biologic construct 21 may be an example of the hybrid construct 150. Actions included in preparing the hybrid subchondral implant-biologic construct 21 at 2030 of can be performed at least partially before, at least partially in parallel with, and/or at least partially after preparing the site at 2020. For example, as one consideration, preparing the hybrid subchondral implant-biologic construct 21 at least partially in advance of preparing the site may allow minimizing or reducing of time elapsed with the site being open or exposed to environment external to the body. As another consideration, preparing the hybrid subchondral implant-biologic construct 21 at least partially after preparing the site may allow sizing or other customizing of the hybrid subchondral implant-biologic construct 21 to account for circumstances encountered during the preparation of the site.

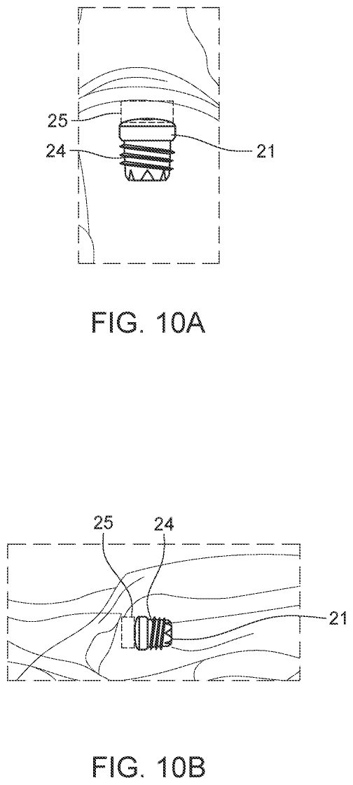

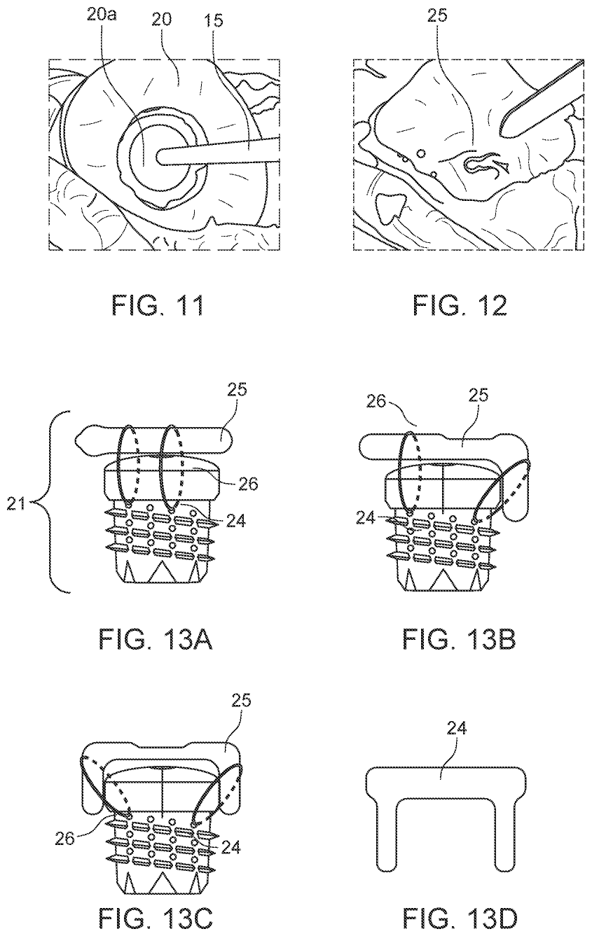

The method 2000 at 2040 can include installing hybrid subchondral implant-biologic construct in the prepared site. For example, this may involve installation in the shoulder joint. Installing may be characterized or affected by factors such as anatomy accessed and/or approach trajectory. Approach trajectories may correspond to examples discussed with respect to FIGS. 14A-14I, for example. Actions in installing may include acts such as described with respect to FIGS. 9, 11, and/or 12 or comparable or other acts for the respective anatomy accessed, for example. Installing may result in placement such as in FIG. 10A or 10B or comparable or other placement for the respective anatomy accessed, for example. As some non-limiting examples, in a case of repair involving a shoulder joint, an anterior approach may be made, a retrograde approach may be made, or a tangential approach may be made to the scapula, the clavicle, the acromion, the sternum, or the humerus, or to a region between the bones, although approaches additionally or alternatively may include antegrade, retrograde, or tangential through, past, or into one or more of the structures noted in FIG. 1A. As one example, in a case of repair involving the AC joint, an approach may be made past the superior or inferior acromioclavicular ligaments and into the clavicle and/or the acromion.

The method 2000 at 2050 can include facilitating treatment of tissue proximate the site. For example, this may involve facilitating treatment of tissue proximate and/or in the shoulder joint. Acts at 2050 may be performed at least partially before, at least partially in parallel with, and/or at least partially after any of the other actions or portions of the method 2000. The hybrid subchondral implant-biologic construct 21 or acts associated therewith may be utilized in conjunction with other surgical techniques or interventions or may otherwise be configured to be beneficial to healing or treatment of tissue proximate the site. As an illustrative example, in a case of repair involving the shoulder 100, placement of the hybrid subchondral implant and biologic construct may augment removal of bone spurs on the acromion or clavicle, or be performed concomitantly with AC capsular ligament repair or reconstruction, CA ligament repair or reconstruction, glenohumeral capsular ligament repair or reconstruction, labral repair or reconstruction, rotator cuff repair or augmentation, biceps tenodesis, repair, or reconstruction, although facilitating treatment of associated tissue may additionally or alternatively include other actions relative to one or more of the structures noted in FIG. 1A. Acts at 2050 may correspond to actions taken to improve outcomes relating to anatomy accessed at 2010, prepared at 2020, subject to installation at 2040, and/or otherwise proximate to the site and in a position to benefit from the presence of the hybrid subchondral implant-biologic construct 21 or associated structures. Acts performed in preparing the hybrid subchondral implant-biologic construct 21 at 2030 may correspond to actions for facilitating treatment of tissue proximate the site at 2050.

The following discussion of FIGS. 2B-12 relate specifically to methods to reconstruct bone and joint surfaces of a patient diagnosed with a condition such as osteochondral fracture defect, osteochondral disease, post-traumatic arthritis/arthroses, bone edema, subchondral micro-trabecular fracture, trabecular fracture, hypertrophic bone, bone cyst, intralesional osteophyte, damaged bone, subchondral bone defect, defective bone, traumatic fracture defect, degenerative fracture defect, iatrogenic fracture defect, iatrogenic causes, osteochondritis dissecans, osteochondritis, osteochondropathy, impingement/instability/arthroses/malalignment/dislocation/subluxation, loose body, Avascular necroses, stress fracture, osteonecrosis, osteopenia, osteoporosis, neoplasm, bone tumor, bone growth, fibrous growth, infection, idiopathic, post-traumatic disease, fracture/fusion/osteotomy, non-union, mal-union, delayed union, stress fracture, insufficiency fracture, pathologic fracture, bone edema micro-trabecular fracture, or chondral defect as exemplary procedures. Additionally or alternatively, procedures can address conditions specifically related to the shoulder, such as AC separation, AC instability, distal clavicle osteolysis, ACJ arthroses/arthritis, post-traumatic arthritis, clavicle fracture, scapular fracture, floating shoulder, etc. . . . It is understood that these procedures and methods can be successfully applied to other human body joints (such as other joints associated with the shoulder and thus should not be limited solely to the surgical treatment of aforementioned conditions. For example, relevant human body joints (and associated bones) may include, but are not limited to, the AC joint, SC joint, glenohumeral joint, or scapulothoracic joint. In various instances, methods for a given joint may be performed to supplement and/or augment other procedures that may be performed on the joint and/or nearby tissue. Examples of adjacent procedures that can occur while an implant is inserted into a joint structure can include fracture repair, fusion, osteotomy, spur or osteophyte removal, chondroplasty, microfracture, abrasionplasty, debridement, LB removal, ligament or capsule repair or reconstruction or advancement, tendon tenodesis/repair/reconstruction. Other examples may be also be appreciated from other discussion herein.

FIG. 2B is a pre-surgical perspective view of a shoulder 10 of a patient according to some examples. Patient history, subjective reporting, and objective evaluations are all clinical risk factors for subchondral insufficiency.

Referring to FIG. 1B, the subchondral bone 10 lies directly beneath the calcified cartilage 5. The subchondral bone 10 can include a bone plate 10A and subchondral trabecular bone 10B. The bone plate 10A is porous and contains channels which allows access to the articular cartilage by arteries, veins, and nerves from the subchondral trabecular bone 10B.

Arising from the subchondral bone plate 10A are the supporting trabeculae, which comprises subchondral trabecular bone 10B, together with deeper bone structure. Subchondral trabecular bone 10B exerts important shock-absorbing and supportive functions in normal joints and may also be important for cartilage nutrient supply and metabolism. Relative to the subchondral bone plate 10A, subchondral trabecular bone 10B is more porous and metabolically active, containing blood vessels, sensory nerves, and bone marrow. Subchondral bone 10 is a dynamic structure and adapts to the mechanical forces imposed across the joint. Mechanical stress also modifies the contour and shape of subchondral bone 10 by means of bone modeling and remodeling.

The articular cartilage 4 covers the subchondral bone 10 and helps to maintain the normal chemical balance and function within the joint. It contains both superficial non-calcified 4 and deep calcified cartilage 5 which are separated by the tidemark. The tidemark provides a transitional structure between the soft cartilage 4 and hard cartilage 5. This functional unit forms the osteochondral junction. The osteochondral junction is complex and includes a deeper layer of non-calcified cartilage 4, the tidemark, calcified cartilage 5, the cement line, and subchondral bone 10. Any alteration of any one of the components will alter the functions of any of the other parts. All the components work in concert with each other. Subchondral bone 10 provides a supportive platform for the overlying articular cartilage 4 and distributes the load in an appropriate manner. When there is degenerative and articular joint destructive disease 3 present there is a significant increase in the load that is transmitted through the articular cartilage 4 to the subchondral osseous structures 10.

The sequela of subchondral insufficiency syndrome begins when the functional alignment and joint motion become out of synch and dysfunctional. The pathologic process of bone loss develops as the unstable articular surfaces track unevenly. This functional abnormality leads to joint space narrowing, degeneration, and thinning of the articular cartilage 4. This leads to a progression of the biomechanical imbalances and the destructive changes 3 progress.

It is important to note that all components of the articular, sub-articular/transitional and subchondral structures are functionally interdependent. Any alteration in structure of any component will alter the functions of any other component, and all components work in concert with each other.

When degenerative and articular joint destructive disease is present there is significant increase in load that is transmitted through the articular cartilage 4 to the subchondral osseous structures 10.

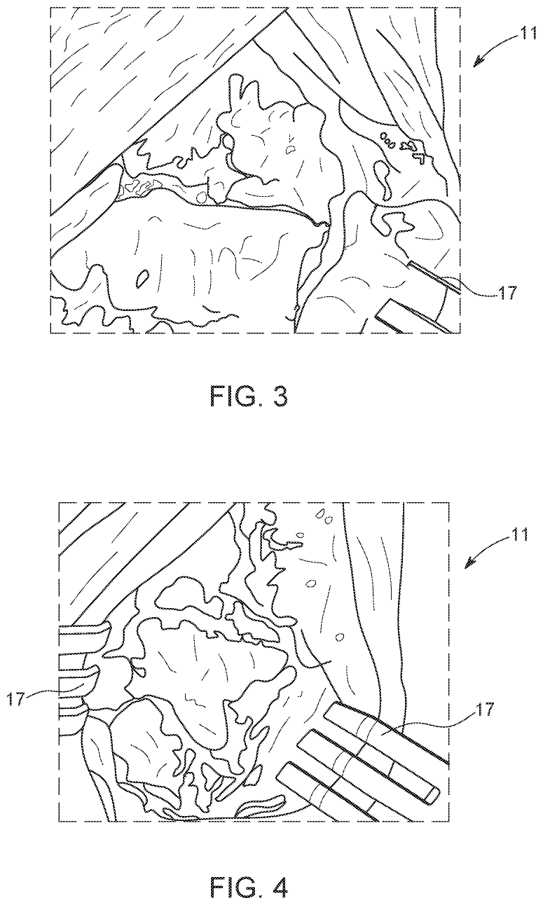

FIGS. 3 and 4 are top views showing open surgical access to a shoulder joint 11 of FIG. 2B. For example, an incision (e.g., a dorsal incision may be made (e.g., along cut line C), and one or more retractors 17 may be used to pull back tissue (e.g., skin, soft tissue, tendon, capsule) and expose underlying elements of the joint (e.g., cartilage and/or bone, such as the acromion, clavicle, scapula, humerus, etc.

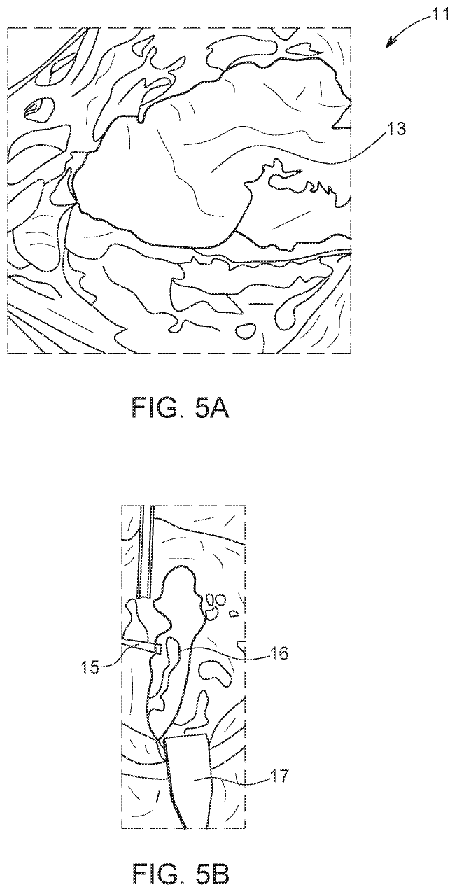

FIG. 5A is a perspective view of a hypertrophic bone 13 with degenerative erosive articular joint disease in the joint 11.

FIG. 5B is a perspective view of a guide pin 15 being advanced toward a defect. The guide pin can be placed in the center of the defect based on perspective imaging or clinical complaints. Additionally, or alternatively, an augmented reality program, such as Osteochondral Augmented Reality (OAR), can process and convert preoperative radiologic data (e.g., MRI, CT, XR, bone scan, etc.) or bone and joint activity imaging/information to augmented reality. The augmented reality can assist a surgeon via orthoscopic or imaging glasses in placing the guide pin accurately in the center of the defect. The center of the defect is approximated by reversibly attaching the guide pin 15 to the subchondral bone 10. The distal end of a sizing instrument is positioned over the defect along the guide pin. The size of the defect is recorded to match a sized hybrid subchondral implant-biologic construct. The sizing instrument can be removed over the guide pin so that a canulated countersink instrument can then be selected and inserted over the guide pin. The countersink should have a diameter substantially matching the measured diameter of the defect.

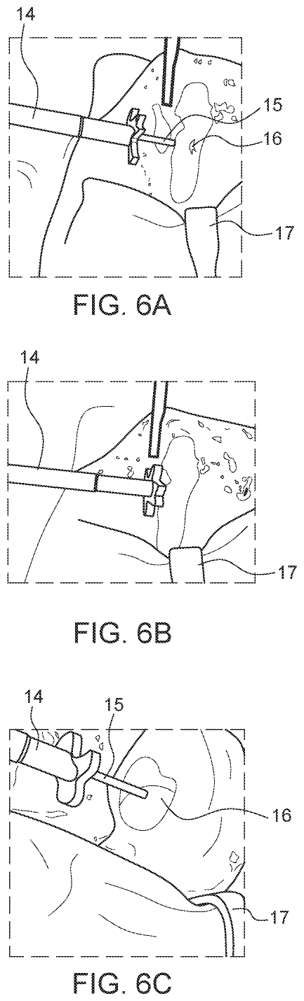

FIG. 6A is a perspective view of a cannulated countersink 14 being advanced along a guide pin 15 toward a defect 16 while a retractor 17 keeps the skin away from the surgical site to aid visualization. In FIG. 6B, the cannulated countersink 14 is in contact with the defect 16. The countersink 14 is then pressed into the defect and rotated to remove tissue. This creates a countersink area (i.e., a partial hole) in the defect 16 surrounding the guide pin 15. The countersink area surrounding the guide pin 15 near the defect 16 is clearly shown in FIG. 6C. Here, the cannulated countersink 15 is being withdrawn from a defect 16 along the guide pin 15.

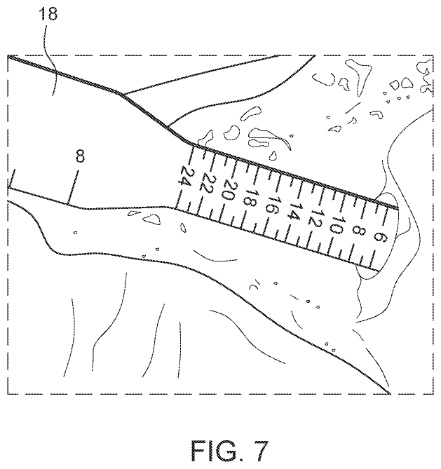

FIG. 7 shows a ruler 18 used to measure the cartilage depth post debridement of diseased articular cartilage to ensure proper placement of the hybrid subchondral implant-biologic construct.

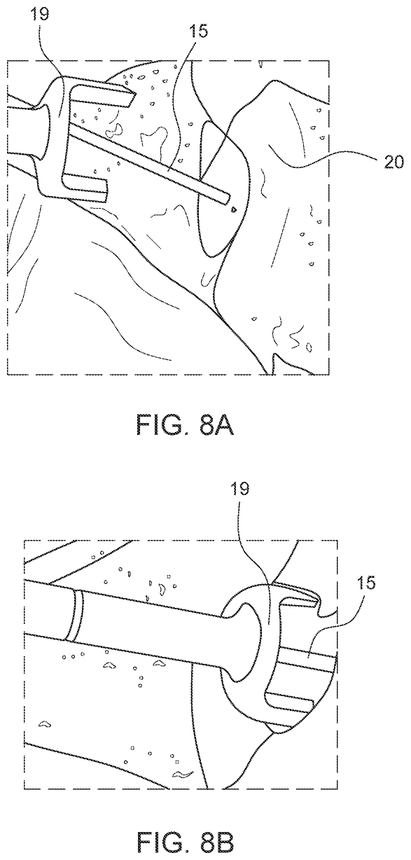

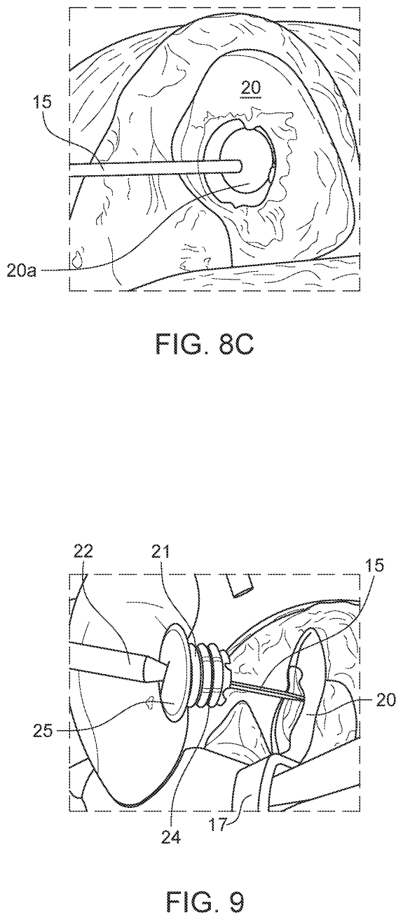

As shown in FIG. 8A, a cannulated coring reamer 19 (also known as a circular osteotome tool) is inserted over (and advanced along) the guide pin 15. As shown in FIG. 8B, the coring reamer 19 is lowered and rotated toward the center of the counter sink hole to create a circular cut and remove a circular portion of the subchondral bone 20 (e.g., to define an annular area) while preserving a central post of subchondral bone in a substantially undisturbed native state (e.g., positioned within a central area bounded by the annular area). An example of the resulting central post of subchondral bone 20 is shown in FIG. 8C. Operation of the reamer 19 can include use of a proprietary double-fluted circular osteotome tool, for example. The reamer 19 may be used to sharply cut bone and rapidly clear bone debride, so as to not impact the prepared bone, thereby encouraging bleeding and/or maximal bleeding. The reamer 19 can be used to remove diseased or hypertrophic bone. The operation of the reamer 19 may also relieve intraosseous pressure, which may be a source of pain. Thus, operation of the reamer 19 may be a portion of the treatment (e.g., relieving pain by relieving pressure) and is not limited to solely preparing the subchondral bone 20 for installation of structure relative to the subchondral bone 20.

FIG. 8C is a perspective view after a cannulated coring reamer 19 has removed an outer circular portion of the subchondral bone 20 at a defect while preserving a central post 20a of subchondral bone 20 in a substantially undisturbed native state. As shown by example in FIG. 8C, the guide pin 15 can remain in alignment with the central post 20a of subchondral bone 20 after the cannulated coring reamer 19 has been removed along the guide pin 15.

FIG. 9 is a perspective view of a hybrid subchondral implant-biologic construct 21 being advanced over the guide pin 15 with a driver instrument 22. The hybrid subchondral implant-biologic construct 21 may be prepared utilizing techniques and/or methods described elsewhere herein, such as with respect to FIGS. 13A-13F and/or FIGS. 15A-15L. The construct 21 can include a subchondral implant 24 and a biologic member 25. The biologic member 25 (which may alternatively be referred to as a biologic construct 25) may correspond to a graft (e.g., a biologic-based graft) and/or some flexible member that includes at least some biologic material (e.g., wholly formed of collagen and/or other biologic material, or formed of biologic material combined with polymer or other material). The subchondral implant 24 can include structure suitable for securing relative to subchondral bone 10. Among other functions, the subchondral implant 24 may provide a suitable infrastructure for supporting and/or positioning the biologic member 25. The construct 21 (e.g., in the portion formed by the subchondral implant 24) may have threads configured to screw the construct 21 into the bone at the hole prepared by the coring reamer. Screwing in or otherwise inserting the construct 21 may position the construct 21 on top of the central post of subchondral bone 20.

FIG. 10A is a radiographic image showing alignment of a joint portion and the implanted hybrid subchondral implant biologic construct 21 in a vertical plane (e.g., a coronal or transverse plane) with the articular defect repaired and the subchondral insufficiency microfractures stabilized according to embodiments of methods to reconstruct bone and joint surfaces according to some examples. FIG. 10B is a radiograph showing alignment of the joint portion and construct 21 in a horizontal plane (e.g., a sagittal plane). A generalized dashed line annotation is super-imposed on each image in FIGS. 10A and 10B to show a general area in which the biologic member 24 portion of the construct 21 may be positioned in the fully installed state. The biologic member 24 may be suitably positioned on account of being coupled with the implant 25 that is secured at a suitable position within the bone. The biologic member 24 may be positioned to be at least partially adjacent a boundary line of the bone formed by adjacent portions of the bone and/or defined by a shape of the bone prior to installation of the construct 21. For example, the biologic member 24 may be positioned to be terminate beneath, at, and/or over the boundary line. The biologic member 24 may be suitably positioned to facilitate healing and/or treatment of cartilage and/or other tissue of the joint, for example.

FIG. 11 shows subchondral bone 20 prepared to receive a hybrid subchondral implant-biologic construct 21 in a fully implanted state in the subchondral bone 20 to stabilize an insufficiency fracture or defect. The subchondral bone 20 in the prepared state may include a central post 20a (e.g., preformed by the coring reamer 19 as discussed above), and the central post 20a may be engaged with a guide pin 15 to facilitate guiding of the contruct 21, for example. As shown in FIG. 12, a hybrid subchondral implant-biologic construct may be implanted with the biologic member 25 suitably placed to approximate or juxtapose the articulating surface of the joint through the installation of the construct 21. To treat osteochondral disease of the patella, a single subchondral implant-biologic construct 21 or multiple constructs can be implanted. In some examples, less than 100% of the osteochondral disease can be treated.

FIGS. 13A-13F are cross-sectional diagrams depicting various orientations and methods to fasten a subchondral implant 24 and biologic construct 25 of a hybrid subchondral implant-biologic construct 21 together according to some examples. The subchondral implant 24 or the biologic construct 25 may be bio-printed or customized to a specific anatomy of a patient. For example, the subchondral implant 24 or the biologic construct 25 can be customized to radii of curvature in multiple planes of a subchondral bone. The implant 24 and construct 25 may be fastened together by one or more fasteners 26. In the depicted views, the fastener are represented as sutures, although staples, adhesive, hook and loop fasteners, clamps, belts, screws, magnets, tacks, rivets, or other fastening means or structures may be utilized. In some embodiments, the fasteners may engage and/or extend through fenestrations of the subchondral implant 24. In some embodiments, the biologic construct 25 may be a graft. In FIG. 13A, the biologic construct 25 is shown atop the subchondral implant 24, while in FIG. 13B, the biologic construct 25 is shown with at least one portion atop the subchondral implant 24 and at least one other portion along a lateral side of the subchondral implant 24. In FIG. 13C, the biologic construct 25 is shown with at least one portion atop the subchondral implant 24 and multiple other portion along different lateral sides of the subchondral implant 24. In FIG. 13D, the implant 24 is in the shape of a staple, such as for tangential insertion as shown in FIGS. 14E and 14F, for example. One of several methods contemplated to deploy the construct 25 is shown in FIGS. 13E and 13F. Here, the construct 25, which may be a graft, rolls or folds up over a trailing end of the implant 24 so the ends 28a, 28b of the construct 25 do not extend beyond the diameter or other greatest dimension of the implant 24 (e.g., remain within a space bounded by reference lines 27 extending axially from opposite ends of a diameter or other greatest dimension of the implant 24). In this manner, the construct 25 may avoid interfering with retrograde insertion (e.g., along direction 29A). The ends 28a, 28b may be maintained in a folded or otherwise retracted state by sutures or other retainer 26 (e.g., which may correspond to a ring or any other structure suitable for retaining the ends 28, 28b in position prior to and/or up to deployment of the construct 25). The retainer 26 may be cut, removed, or otherwise disengaged, which may allow the ends 28a, 28b to be deployed, such as by rotating at arrow 29B or otherwise relocating along the sides and/or top of the implant 24. The retainer 26 may be disengaged prior to, after, and/or in response to removing a driver (e.g., along a direction indicated by arrow 29C). FIG. 13F represents the implant 24 after placement with the retainer 26 released and after the construct 25 is deployed. The construct may be held or secured in place after deployment by new sutures 30 or other fastening means known to those of skill in the art.

FIGS. 14A-14I are cross-sectional diagrams depicting various approaches and orientations to implant a hybrid subchondral implant-biologic construct 21 into a bone 20 according to embodiments of methods to reconstruct bone and joint surfaces. The bone 20 may be part of a shoulder joint. For example, the bone 20 may be a acromion, scapula, clavicle, humerus, sternum, or etc.

The surgical method may involve bone preparation. A countersink reamer can be used to prepare a superficial portion of bone and to remove diseased or hypertrophic bone. A tool (e.g., a double-fluted osteotome tool) can assist in performing a Circular Decompression Osteotomy (CDO). The CDO can involve sharply cutting bone and rapidly clearing bone debride so that the prepared bone is not impacted. The CDO can permit maximal bleeding. The CDO can also reduce intraosseous pressure, which can be a source of pain.

The surgical method may involve inserting the hybrid subchondral implant-biologic construct 21 in through the joint with a guide pin 15 or guidewire inserted perpendicular to the joint defect (i.e., a perpendicular insertion antegrade into joint) of the bone 20, such as illustrated by arrow 32A in FIG. 14A. Alternatively, the surgical method may involve inserting the hybrid subchondral implant-biologic construct 21 tangentially, transversely, or non-parallel to a longitudinal axis of the joint. For example, insertion may be performed at an approach trajectory that is a 90 degree angle from a longitudinal axis of the joint and bone 20 (e.g., such as shown by arrow 32E in FIG. 14E to arrive at a position shown in FIG. 14F). With this approach, a socket driver 31 may be used to adjust the height of the hybrid subchondral implant-biologic construct 21 in the bone 20 with respect to the joint after the hybrid subchondral implant-biologic construct 21 is seated longitudinally. As one example, the socket driver 31 may approach along a longitudinal axis toward the end of the construct 21 and rotate about the axis, such as depicted by arrows 32Ea and 32Eb, respectively. As another example, the socket driver 31 may function similarly to a crescent wrench and be capable of engaging the construct 21 along a periphery rather than along the longitudinal axis of the construct 21, such as undergoing a same or similar approach trajectory as the construct 21 and then rotating as depicted by arrows 32Ec and 32Ed, respectively.

The surgical method may involve a non-perpendicular approach trajectory. For example, as may be best seen by way of example in FIGS. 14B-14D, the surgical method may involve inserting the hybrid subchondral implant-biologic construct 21 along the guide pin 15 at an angle ranging from between about 20-70 degrees (or 20-90 degrees) from a longitudinal axis of the joint (e.g., as illustrated by arrow 32B in FIG. 14B), and subsequently pivoting the hybrid subchondral implant-biologic construct 21 toward the longitudinal axis of the joint (e.g., as illustrated by arrow 32C in FIG. 14C) and/or otherwise repositioning until the hybrid subchondral implant-biologic construct 21 is positioned in a correct anatomic position (e.g., such as the state shown in FIG. 14D). With this approach, a socket driver 31 may be used to adjust the height of the hybrid subchondral implant-biologic construct 21 in the bone 20 with respect to the joint after the hybrid subchondral implant-biologic construct 21 is seated longitudinally. Examples depicted in FIGS. 14B-D involve methods to pivot the implant in, or the implant can be dropped in horizontally as depicted in FIG. 14E. Alternatively, the hybrid subchondral implant-biologic construct 21 may be inserted in a retrograde fashion along the guide pin 15, such as shown by way of example by the arrow 32H in FIG. 14H. FIG. 14G shows yet another orientation where the guide pin 15 is curved and the hybrid subchondral implant-biologic construct 21 is inserted along the guide pin 15 curved toward the right or left. FIG. 14I illustrates one hybrid subchondral implant-biologic construct 21 in each bone 20 on either side of a joint, although any combination of one or more than one hybrid subchondral implant-biologic construct 21 may be used, such as including the same or different types (which may be installed in similar or different approach trajectories) in bones 20 on either side of a joint and/or including one or more than one in a given bone 20.

The surgical method may involve a trajectory through the bone. For example, as may be best seen by way of example in FIGS. 14J-14K, the surgical method may involve removing a column 33 of bone 20 in retrograde from directly beneath the joint, such as depicted by arrow 32J in FIG. 14J. The column 33 may be removed for example with a coring reamer, to access the involved subchondral bone and form a subchondral cavity 34, e.g., prior to final preparation for the hybrid subchondral implant-biologic construct 21. The method may further involve putting the construct 21 in retrograde from directly beneath the joint, such as into the cavity 34, e.g., as depicted by arrow 32K in FIG. 14K. The method can further involve placing the construct 21 into the subchondral bone, such as depicted in FIG. 14L. The method can further involve placing the column 33 of bone 20 into the chamber of the construct 21 from below, such as depicted in FIG. 14M. Alternatively, as may be best seen by way of example in FIGS. 14N-14Q, the driver instrument 22 may come from a retrograde trajectory (such as depicted by arrow 32N in FIG. 14N), but the construct 21 will engage the driver within the joint (e.g., FIG. 14O), and the driver instrument 22 will advance the implant from the top down with the driver instrument 22 inserted from within the construct 21 from a retrograde direction (e.g., FIGS. 14P and 14Q).

Methods described herewith may be used to treat conditions of the shoulder in addition to or in lieu of AC instability, AC separation, etc. For example, the shoulder joint may be reconstructed with a hybrid subchondral implant-biologic construct 21, that reconstructs both the subchondral bone and joint surface.

In this method, the biologic construct may be a graft of the same size, larger than, or asymmetric with respect to the subchondral implant. In this method, the biologic construct may be attached to the top or sides of the subchondral implant, such as shown in FIG. 13E. In this method, the edges of the biologic construct may be tucked down over the sides of the implant, such as covering and/or extending through the wall/side fenestrations, such as to the position shown in FIG. 13F. This may be done after implantation, for uncontained defects. With this approach, a socket driver may be used to adjust the height of the hybrid subchondral implant-biologic construct 21 in the bone 20 with respect to the joint after the hybrid subchondral implant-biologic construct 21 is seated longitudinally.

As shown in FIG. 13E, sutures, staples, or the like may be inserted through the central driver, which folds the graft up like the petals of a flower, to allow retro-grade insertion. Upon seating of the implant, the suture or staple is released, unfolding the biologic construct as shown in FIG. 13F.

The surgical method may involve a flexible guide pin, and flexible reamers and driver, to allow the subchondral implant to come into the joint at one angle or trajectory, and then turn to follow the flexible guide pin for final seating. The reconstruction may involve a single or multiple hybrid subchondral implant-biologic constructs. The subchondral bone and joint reconstruction may involve more than one separate hybrid subchondral implant-biologic construct, for example, placed adjacent to each other following the radius of curvature of the subchondral bone or otherwise arranged adjacent and/or relative to one another. A single biologic surface can be affixed to multiple implants separately. A flexible guide pin may be placed in such a manner that the implant will follow the guide pin to the aperture of the defect, then a retrograde driver or socket driver may be attached to the implant to allow final seating.

In some examples, reaming and bone preparation can be performed retrograde and a driver can be placed through bone into the joint. The implant can be docked to the driver from within the joint. The implant can be delivered to the driver at the joint via a flexible guidewire or suture cable. A surgical method including the mini-anchors may involve inserting the hybrid subchondral implant-biologic construct 21 at an angle ranging from between about 20-70 degrees relative to lateral fenestrations to allow the mini-anchors or polyaxial sutures to implant the construct 21.

The reconstruction may involve separate hybrid subchondral implant-biologic constructs, such as with a single surface of a biologic construct affixed to the subchondral implants separately (e.g., a one-to-many arrangement with a biologic construct extending to be at least partially over more than one implant). The surface or the entirety of the subchondral implant may be bio-printed or customized to the individual patient's specific anatomy and radii of curvature in multiple planes. A radius of curvature of the subchondral implant can be customized to be equivalent or slightly smaller than the radius of curvature associated with the joint measured for the patient. For example, the radius of curvature of the subchondral implant can fall within a range of 50% to 100% of the radius of curvature associated with the joint of the patient. The radius of curvature of the joint can vary across multiple planes and a design of the subchondral implant can take this variation into account. For example, an aspect ratio associated with the subchondral implant can made more shallow for joints that are difficult to access. The method may involve immediate post-operative weight-bearing and range-of-motion without restrictions.

It is contemplated that such methods may involve putting a slit through the biological construct to allow the driver to access the subchondral implant. The slit may be configured to close after the driver is removed. A size of the slit and/or a material of the biological construct may facilitate such post-removal closure. For example, the biological construct may be formed of sufficiently flexible material that boundaries of the slit can flex away from each other when the driver is inserted into and/or is present in and/or through the slit. The material of the biological construct may also be sufficiently resilient so as to exhibit a memory and/or otherwise return toward a state in which the boundaries of the slit are in contact with one another or spaced apart by a negligible distance so as to be in a closed state after the driver is removed.

In various examples, the hybrid subchondral implant-biologic construct 21 may be formed independent of the driver instrument 22 and be subsequently engaged with the driver instrument 22 for installation. Alternatively, in some examples, the hybrid subchondral implant-biologic construct 21 may be formed at least in part around the driver instrument 22. FIGS. 15A-15L illustrate operations that may be utilized to prepare the hybrid subchondral implant-biologic construct relative to a driver instrument 22.

As represented in FIG. 15A, material for the biologic construct 25 may be formed into a suitable shape and size. For example, this may include punching or cutting from a sheet 35 or other supply of suitable material (e.g., which may be material for a graft or other suitable material). A suitable forming instrument 36 may be utilized, such as a blade, punch, shaping spatula, or other tool. The size of the biologic construct 25 may be selected based on a size of the subchondral implant 24 to be utilized and/or a size of a defect or other surgery site feature.

As represented in FIG. 15B, a slit 37 may be formed through the biologic member 25. The slit 37 may be formed by a piercing instrument 38, which may be the same as the forming instrument 36 or may be any other suitable tool for piercing the material of the biologic construct 25 to form the slit 37 extending therethrough. The slit 37 may be formed to have any suitable shape or geometry. Non-limiting examples include shaped with crossing cuts (such as in an X-shape); shaped as a single line; shaped as an intersection of 2, 3, 4, or more lines; jagged; smooth; or any other shape to facilitate engagement with the driver instrument 22 (e.g., for permitting the driver instrument 22 to pass through the slit 37 and/or to cause the slit 37 to close upon removal of the driver instrument 22). The piercing instrument 38 may be specially shaped to match and/or impart the shape of the slit 37, or may be suitably contoured to form different segments of the slit 37 in response to different piercing motions performed by the piercing instrument 38.

As represented in FIG. 15C, the biologic construct 25 can be mounted on the driver instrument 22. For example, the slit 37 in the biologic construct 25 can be passed over around an outer periphery of the driver instrument 22 and/or the driver instrument 22 can be inserted through the slit 37. The fit of the slit 37 for the driver instrument 22 can be checked and the the slit 37 modified (e.g., enlarged or at least partially formed back together) if needed to improve fit. The biologic construct 25 may be arranged so that a driving interface 22A of the driver instrument 22 extends below the biologic construct 25. In some examples, the biologic construct 25 can be a graft and the slit 37 can be formed in the graft, which can be mounted on the driver instrument 22.

As represented in FIG. 15D, the subchondral implant 24 can engage with the driver instrument 22. For example, the driving interface 22A of the driver instrument 22 may engage a mating interface of the subchondral implant 24. The subchondral implant 24 may be positioned adjacent the biologic construct 25 on the driver instrument 22 upon engagement with the driver instrument 22.

The biologic construct 25 and the subchondral implant 24 may be secured to one another along the driver instrument 22 by any suitable technique. As one example represented starting in FIG. 15F, a suture 40 with needles or other guides 42 attached at each end may be utilized. As represented in FIG. 15G, the guides 42 may be inserted through a top of the biologic construct 25 and also through the subchondral implant 24 (e.g., via fenestrations in the top of the subchondral implant 24). As represented in FIG. 15H, the guides 42 may accordingly extend out through a bottom of the subchondral implant 24, such as in a position suitable for gripping and continuing to pull. Pulling the guides 42 from an underside of the subchondral implant 24 can draw an upper loop of the suture 40 against the biologic construct 25, for example. The guides 42 may be pulled from the underside of the subchondral implant 24 to a suitable position for manipulating to knot or otherwise secure the suture 40. For example, as represented in FIG. 15I, the suture 40 may be knotted with whatever knot is preferred by the surgeon. Loops or other segments of the suture 40 may be pushed by a knot pusher 44 or other suitable tool in the process of completing the knot. The knot pusher 40 may facilitate locating the knot in a chamber 46 defined on an underside of the subchondral implant 24, for example. The knot may be secured along an underside interior of the subchondral implant 24. As represented in FIG. 15I, the suture 40 may be cut a suitable distance from the knot. For example, the suture 40 may be trimmed to provide suitable tails to reduce a possibility of the knot unraveling and/or may be trimmed so that the cut ends of the suture do not extend out of the chamber 46 after cutting. A set of scissors, a blade, or another suture cutter 48 may be used to cut the suture 40. Overall, the knotted suture 40 may secure the biologic construct 25 atop the subchondral implant 24.

Other processes may also be utilized to secure the biologic construct 25 relative to the subchondral implant 24. As one other example represented in FIG. 15E, a mulberry knot or other suitable form of stopping knot may be formed in a suture 40 to act as a stop for a trailing end of the suture 40. A leading end of the suture 40 may be guided by a needle 42 or other guide structure down through a top of the biologic construct 25 and to a suitable location for coupling with the subchondral implant 24, e.g., similar to operations described with respect to FIGS. 15H-15J for imparting a stopping knot on the underside to secure the biologic construct 25 to the subchondral implant 24 via the suture 40. Generally, approaches from FIG. 15F and FIG. 15E may differ with respect to whether knots are located on both top and underside or only on underside of the subchondral implant 24. Other alternatives may include routing a leading end of the suture 40 to any other suitable anchoring feature of the subchondral implant 24 (e.g., extending to projections or along an underside of the subchondral implant 24.

As represented in FIG. 15K, securing the biologic construct 25 with the subchondral implant 24 may include or be supplemented with conforming the biologic construct 25 to the subchondral implant 24. For example, material of the biologic construct 25 may be pressed down and/or sculpted to reach the periphery of the subchondral implant 24 or otherwise conform to a suitable form factor for the procedure. Conforming the biologic construct 25 may include adjusting and/or positioning the biologic construct 25 to have any configuration discussed with respect to FIGS. 13A-13F or any other suitable position relative to the subchondral implant 24, for example. Similarly, securing the biologic construct 25 may include securing by any arrangement discussed with respect to FIGS. 13A-13F or any other suitable arrangement of sutures or other structures for coupling with the subchondral implant 24. Coupling the biologic construct 25 with the subchondral implant 24 can produce the hybrid subchondral implant-biologic construct 21 in a preassembled state precoupled with the driver instrument 22, such as represented in FIG. 15L. Preassembled parts may be incorporated into a surgical assembly. For example, a resulting surgical assembly may be configured for use in a surgery to be performed relative to a shoulder joint, such as for intervening in subchondral bone therein to ameliorate one or more defects. In varying examples, the surgical assembly may include the driver instrument 22 and the hybrid subchondral implant-biologic construct 21 and/or more, fewer, and/or other components, which may include others described herein.

The hybrid subchondral implant-biologic construct may be configured in various ways to facilitate accessing and inserting the implant into a specific bone. The biologic construct 25 may be attached to the top of the subchondral implant 24, but a peripheral attachment may be performed after the subchondral implant 24 is fully seated in the bone, For tangential or angled insertion, the hybrid subchondral implant-biological construct may be configured to contain notches that occupy a greater percentage of threads on the subchondral implant 24. The subchondral implant 24 may resemble a staple, for translational insertion, for example. The aspect ratio of the hybrid subchondral implant-biological construct may be configured to be shallower for joints that require difficult or tortuous access. The hybrid subchondral implant-biological construct may be configured to have fenestrations. The angle of lateral fenestrations may be between about 20-70 degrees to allow insertion of fasteners polyaxially to secure the implant into a bone.

In some examples, the biologic construct 25 can be a graft. The graft can provide reinforcement of soft tissues in an area of weakness at or near an implant site. The graft can provide a complete repair of deficient or missing osteochondral soft tissue. Alternatively, the graft can be a partial-repair, or soft tissue nidus, and allow further healing to a healing biologic surface. Structural properties of a graft can be similar or improved relative to native tissue. In some examples, the graft can include collagen, a polymer material component, or a mixture of both. Examples of the polymer material component can include polycaprolactone (PCL) or a mineral-polymer composite of with hydroxyapatite (HA) with PCL. The HA-PCL composite can be designed to encourage chondrous or fibrochondrous tissue ingrowth.

The graft can include a percent absorption profile that allows the graft to maintain 70-90% strength for at least 6-18 months post-implantation. The subchondral implant 24 can include studs with microbarbs. The microbarbs can secure the graft to the subchondral implant 24. The graft can have a porosity consistent with pore sizes of 100-700 micron diameters. The porosity of the graft can encourage chondrous or fibrochondrous tissue formation. The graft can include a fiber orientation (e.g., random) that can encourage collagen and matrix formations that can resist shear stresses. The hybrid subchondral implant-biologic construct can include porous biological material impregnated with matrix-promoting substances or serve as a scaffold for progenitor cells or include both porous biological material impregnated with matrix-promoting substances and serve as a scaffold for progenitor cells.

The graft can be affixed to the subchondral implant 24 to form an implant-graft construct. In some examples, the graft can be reduced onto a top of the subchondral implant 24. A stencil tool can be affixed onto a top of the graft to guide the graft onto the subchondral implant 24 via needle placement. Double-loaded S-Fibre straight needles can be placed through the top of the graft and delivered on an underside of the subchondral implant 24. Affixing the graft to the subchondral implant can involve tying sutures. The implant-graft construct can be inserted into a joint defect to reconstruct or partially reconstruct the joint or to address a defect or osteochondral disease. In some examples, the implant-graft can be inserted via guidewire.

In some procedures, biologic healing properties of the graft can be enhanced by using allograft or autologous concentrated growth factor (CGF). Allograft can be a tissue graft received from a donor. Additionally, these procedures can involve placental tissues, umbilical tissues, or pluripotent cellular materials. CGF can involve a promotion of tissue regeneration with autologous platelet concentrate. These procedures can involve anti-coagulants to encourage bleeding.

In some procedures, the subchondral implant 24 can act as a delivery vehicle for deploying additional materials in a vicinity of the joint. The additional materials can be packed as particulates or as polymer biologic-hybrid cartridges placed in a chamber of the subchondral implant 24. Examples of the additional materials can include antibiotics, chemotherapeutic substances, chondrogenic or osteochondrogenic material, osteogenic material, etc.

In some procedures, material for the graft can include collagen Poly(lactic acid) (PDLA) or other similar polymer scaffolding. A use of collagen PDLA can reinforce or replace deficient or weakened tissue. The graft can include a random superficial orientation of collagen fibers to enhance random collogen and/or tissue growth. Deeper fiber orientation of the collagen can encourage vertical collagen fiber or tissue growth, for bonding to deeper tissues. Pore sizes of the collagen, which can range from 150-500 microns, can encourage hyaline-like matrix formation and chondrogenesis. The method can include a peripheral portion of scaffold tapers, to allow native tissue overgrowth and bonding.

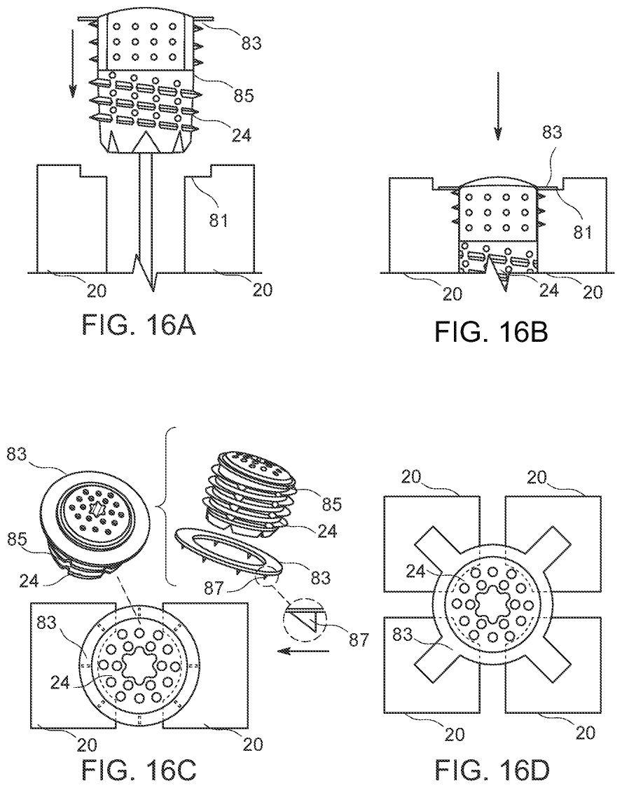

The surgical method may involve inserting the hybrid subchondral implant-biologic construct 21 tangentially, transversely, or non-parallel to a longitudinal axis of the joint. For example, insertion may be performed at an approach trajectory that is a 90 degree angle from a longitudinal axis of the joint in between multiple bones, such as the two bones 20 on opposite sides of the joint shown in FIG. 16A-D. Such tangential approach may be useful to facilitate fusing of bones along a joint.

FIG. 16A shows a side view of an implant 24 (e.g., which may be part of the implant-biologic construct 21 or implemented independently) being guided tangentially towards the joint. The implant may be guided toward a site prepared per operation 2020. The preparation at 2020 may include preparing a site of subchondral bone 20 at the joint by removing damaged tissue in and/or between the subchondral bone of multiple bones along the joint so as to form a bore between the multiple bones along the joint. For example, reaming may be performed to remove damage (e.g., in bone and/or cartilage) in the joint. A countersink ledge 81 may be formed around the bore, such as to provide a suitable location for seating structure extending from the implant. FIG. 16B depicts a side view of the implant 24 positioned within the joint and fusing the two bones 20 on opposite sides of the joint together. FIG. 16C shows a top view of the implant 24 positioned within the joint and fusing the pair of bones 20 together. Upon installation in the bore, the implant may engage the bones, such as via threads on an exterior of the implant and/or such as by receipt of some portion of native bone within a central cavity of the implant. For example, the implant is shown with a cylindrical body 85 having a body outer diameter sized and configured to engage portions of multiple bones along a joint for fusing of the joint. The implant additionally may include an inner diameter sized to receive portions of bone (e.g., which may include some native retained bone that the internal chamber of the implant can receive and/or retain). In some embodiments, the internal chamber of the implant can be packed with healthy bone (e.g., from bone local to the joint or obtained from a cadaver or elsewhere). Packing with healthy bone may facilitate fusing across the joint (e.g., which may include fusing through fenestrations in the joint.

The implant further can include other structure to facilitate fusion. As one example, the implant can include a washer flange 83 laterally extending from the cylindrical body 85 away from the body outer diameter and to a washer outer diameter sized to engage the bones along the joint for transferring load thereto. The washer flange 83 is shown in exploded form in FIG. 16B to depict that the washer flange may be formed in a washer coupled with the cylindrical body 85, although the washer flange may be monolithic with the cylindrical body. In use, the washer flange may sit within the countersunk portions of the bones when installed. Generally, fusing with structure received in a countersink or otherwise recessed into the joint may allow fusion without prominent hardware that may extend upward from the native surface of the joint. This may avoid painful contact with tendons or other soft tissue with outwardly extending structure (such as may otherwise occur if a plate or other prominent structure is present over the position of the native surface of the joint).

The washer flange 83 may include at least one spike 87 arranged to extend into bone. The spike may have an angle, bevel, and/or other shape that encourages compression of the bone against the implant and other bone to promote healing and fusion. For example, the spike may be shaped with one amount of incline on one side that may differ from an amount of incline on an opposite side, such as to impart a biasing force on the bone in a direction based on the difference in incline (e.g., such as depicted by the leftward arrow in FIG. 16C). Differing spikes on opposite sides of the joint may be configured to bias the bones toward one another, for example.

The implant can be deployed to fuse multiple bones together. FIG. 16D shows a top view of the implant 24 implanted within a joint and fusing four bones 20 together. Although four bones are depicted in FIG. 16D, the implant 24 may fuse less than or more than four bones. For example, two bones are shown engaged with the implant in FIG. 16C, while four bones are shown engaged by the implant in FIG. 16D, although any other number of multiple bones may be engaged by an implant to facilitate fusing. Moreover, the washer flange 83 is not limited to circular shapes but may extend in any direction to facilitate engagement with bone. For example, in FIG. 16D, the washer flange 83 is shown with some examples of radial extensions that extend away from an exterior of the washer, although any shape of extension or other gripping feature may be implemented.

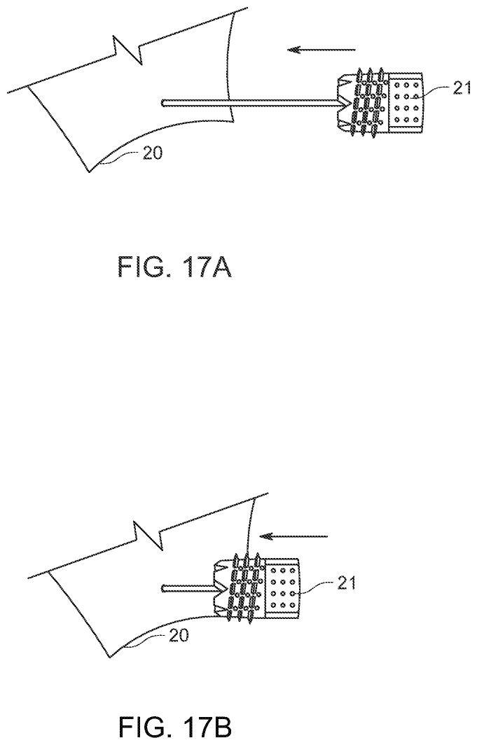

Tangential insertions of the implant-biologic construct 21 can involve insertions into a single bone in some embodiments, such as depicted in FIG. 17A-B by way of example. Tangential insertions may facilitate healing and operation of the joint (e.g., in lieu of fusion) in some embodiments. Tangential insertions may facilitate arrangements in which a side (e.g., a lateral side) rather than an end (e.g., a longitudinal end) of the implant forms a working surface and/or is aligned along a surface of the joint. FIG. 17A depicts a side view of the implant-biologic construct 21 as the construct 21 is guided tangentially towards a single bone 20 associated with a joint. As depicted in FIG. 17A, the joint surface (e.g., surface of a bone facing the joint) can be a concave surface. Alternatively, the joint surface can be convex or flat. In FIG. 17B, the implant-biologic construct 21 is embedded in the bone 20 on a side of the joint surface. For example, upon installation, a lateral side of the implant may be arranged along the joint surface. In some arrangements, the implant-biologic construct 21 may be installed so that a top surface and at least one side surface thereof are arranged along surfaces of the joint.

When the implant-biologic construct 21 includes a graft 24, the graft can be folded onto a joint surface upon insertion of the implant-biologic construct 21 after a tangential approach. This scenario is depicted in FIG. 18. FIG. 18 shows a side view of a graft 24 folded onto a joint surface associated with a tangential insertion process of an implant-biologic construct 21. In FIG. 18, the joint surface is concave, but grafts can be folded onto convex and flat surfaces as well. The graft 25 may be folded onto an inset wall 156, for example, such that such that a first part of the biologic member is received along the inset wall and a second part of the biologic member is received along a top of the trailing portion of the implant 24. For example, in use, with tangential insertion and the biologic member and/or graft arranged at least partially along a side of the implant, the biologic graft may substantially augment the chondral portion 88 of the osteochondral defect or damage and may thereby covert tangential stresses from the joint (e.g., in substantially a left/right direction in FIG. 18) to compressive stresses (e.g., in substantially an up/down direction in FIG. 18) via the implant-graft interface (e.g., and such compressive stresses may be subsequently transferred via the hollow chamber-retained bone interface, and finally through the outer bone-implant interfaces).

In some examples, anchors 91 or mini-anchors can be deployed to secure the hybrid subchondral implant-biologic construct 21. The anchors can be all-suture anchors, polymer anchors, darts, metal anchors, or some combination or mixture thereof. Alternatively, the anchors can be made of a material with hardness greater than that of titanium or of the subchondral implant and threaded for self-drilling. A surgical method including the anchors may involve inserting the hybrid subchondral implant-biologic construct 21 at an angle ranging from between about 20-70 degrees relative to lateral fenestrations to allow polyaxial insertion of the anchors into the fenestrations for implanting the construct 21. Additionally, the surgical method involving the anchors can involve a tack, staple, or sutures. The surgical method involving anchors can involve using a drill with a drill bit for anchor placement. The drill bit can be composed of harder material than the construct 21 and may penetrate the construct 21 at any angle for anchor placement. The construct 21 can include secondary mini-anchors that can be deployed via lateral fenestrations to repair soft tissue adjacent to the joint. An example of a deployment of a secondary mini-anchor is depicted in FIG. 19. A mini-anchor 91 can include a t-shaped attachment 93 (similar to a clothing price tag), such as depicted at left in FIG. 19), a threaded interface 95 (such as depicted at right in FIG. 19), or any other structure that can allow a tether 97 of the mini-anchor 91 to be secured to the construct 21 (such as through the graft 24, as at arrow 98) and/or to surrounding tissue 99 (as at arrow 96). Examples of the adjacent soft tissue can include capsules and ligaments. The t-shaped attachment can be pushed through soft tissue and passed through holes in the construct 21. The secondary mini-anchors can also include threading or can be fastened to the soft tissue using staples or darts. Generally, at least one anchor can be included in engagement with a fenestration through the subchondral implant, and the anchor may be coupled with a tether 97 sized and arranged for coupling with the biologic member 25 and/or for coupling with surrounding tissue 99 at an installation site.

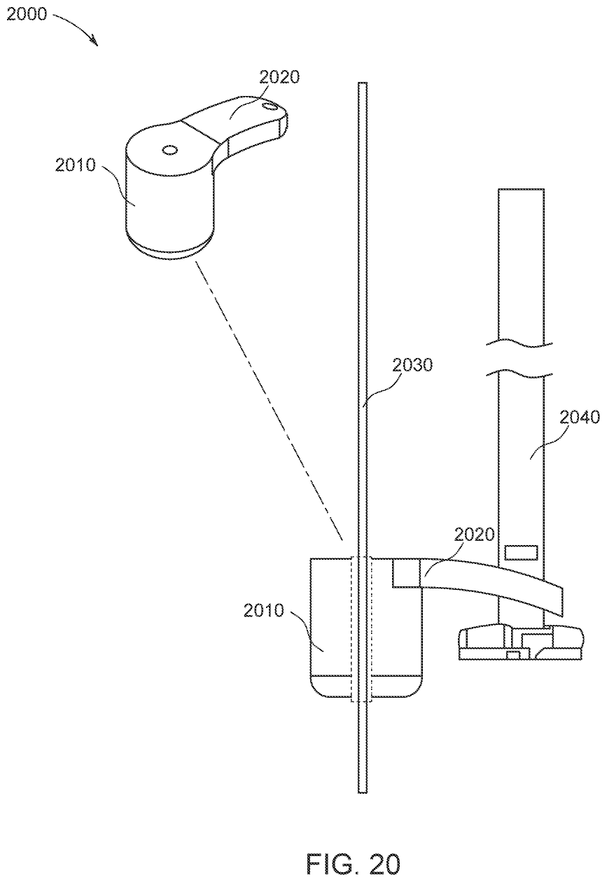

FIG. 20 is a schematic of an alignment attachment 2000 for guiding a secondary tool 2040 according to some aspects of the present application. The alignment attachment 2000 can be a component of a surgical assembly that includes a subchondral implant. The alignment attachment 2000 can include a base 2010 with a lumen sized for engaging a guide wire 2030. The guide wire 2030 can be used to position the alignment attachment 2000 near a joint during a surgical operation. An arm 2020 can extend from the base 2010. Design parameters of the arm 2020 can be based on a particular joint or class of joints. For example, the design parameters of the arm 2020 can include a radius of curvature. A size of the radius of curvature can be based on a size of a native radius of curvature of a surface associated with the particular joint or class of joints. For example, the radius of curvature may be provided in small, medium, and large sizes (and/or in other predetermined sets of general sizes) to generally accommodate curvature of a surface of a joint and/or variations that may be encountered, or the radius of curvature may be tailored to implementation with a specific joint. Generally, the arm 2020 may be formed with a radius of curvature selected to accommodate a curvature of a surface of the joint. In some examples, a radius of curvature of the arm 2020 can allow one operation at a normal orientation at a given position along a contour of a joint and may align other components for operation also at a normal orientation at a different part of a curve or contour along the joint. Design parameters of the arm 2020 can include a length of the arm 2020. For example, the arm 2020 may be implemented with a suitable length so that an implant installed using the guide line will not be interfered with by an implant installed using the secondary tool 2040 as guided by the alignment attachment 2000.

The base 2010 can also include an alignment aperture that can be used to position the secondary tool 2040. The secondary tool 2040 can be used to perform an operation in parallel or adjacent to another operation associated with the guide wire 2030. For example, a first operation involving insertion of a subchondral implant at a first location associated with a joint can occur along the guide wire 2030. The secondary tool 2040 can be involved in a second operation involving an insertion of an additional subchondral implant at a second location associated with the joint. The second operation can occur before, during, or after the first operation during a single surgical procedure. The alignment attachment 2000 may facilitate various operations, which may include, but are not limited to alignment of a reamer to create a countersunk area at the joint (e.g., for a washer or graft), or for installation of a second implant at a precise distance from a first.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1.. A surgical assembly configured for use in a surgery to be performed relative to a glenohumeral or other joint of the shoulder for intervening in subchondral bone therein to ameliorate one or more defects, the surgical assembly comprising:
a driver instrument; and
a hybrid subchondral implant-biologic member construct configured to be installed relative to the glenohumeral or other joint of the shoulder and comprising:
a biologic member having a slit formed therethrough, wherein the slit is arranged so that the driver is extending through the slit, wherein the biologic member is tailored to provide therapeutic effect in the glenohumeral or other joint of the shoulder; and
a subchondral implant engaged with the driver, wherein the subchondral implant is sized and shaped to be received into the subchondral bone of the glenohumeral or other joint of the shoulder, wherein the biologic member is secured to the subchondral implant so that the hybrid subchondral implant-biologic member construct is formed on the driver to facilitate installation into the glenohumeral or other joint of the shoulder by operation of the driver.

2. The surgical assembly of claim 1, wherein the subchondral implant and the biologic member are configured to be installed as the hybrid subchondral implant-biologic member construct over a top of a central post formed by removing a circular portion of the subchondral bone in the glenohumeral or other joint of the shoulder while preserving the central post of the subchondral bone in a substantially undisturbed native state.

3. The surgical assembly of claim 2, wherein the subchondral implant comprises a cylindrical body having a leading portion and a trailing portion, wherein the leading portion defines an open bottom sized for receiving the central post, and wherein the trailing portion comprises an inset wall inset toward a longitudinal axis of the body and away from an outermost perimeter of the body, wherein the inset wall is sized and arranged for receiving the biologic member in an arrangement in which the biologic member is folded such that a first part of the biologic member is received along the inset wall and a second part of the biologic member is received along a top of the trailing portion.

4. The surgical assembly of claim 3, wherein the subchondral implant further comprises at least one prong extending from the inset wall and sized and configured for engaging the biologic member.

5. The surgical assembly of claim 1, wherein the biologic member is retained folded up over a trailing portion of the subchondral implant by at least one suture or other retainer.

6. The surgical assembly of claim 1, further comprising at least one retainer securing the biologic member such that at least one portion of the biologic member is atop the subchondral implant and at least one other portion of the biologic member is along a lateral side of the subchondral implant.

7. The surgical assembly of claim 1, wherein the subchondral implant comprises a cylindrical body having a body outer diameter sized and configured to engage portions of multiple bones along a joint for fusing of the joint, wherein the subchondral implant further comprises a washer flange laterally extending from the cylindrical body away from the body outer diameter and to a washer outer diameter sized to engage the bones along the joint for transferring load thereto.

8. The surgical assembly of claim 7, wherein the washer flange is formed in a washer coupled with the cylindrical body.

9. The surgical assembly of claim 7, wherein the washer flange includes at least one spike arranged to extend into bone and having angle, bevel, and/or other shape that encourages compression of the bone against the implant and other bone to promote healing and fusion.

10. The surgical assembly of claim 1, further comprising at least one anchor in engagement with a fenestration through the subchondral implant, the anchor coupled with a tether sized and arranged for coupling with the biologic member and/or for coupling with surrounding tissue at an installation site.

11. The surgical assembly of claim 1, further comprising:
a guide wire alignable with the driver instrument; and
an alignment attachment comprising a base defining a lumen sized for engaging the guide wire, the alignment attachment further comprising an arm extending from the base and comprising an alignment aperture positioned for receiving a secondary tool therethrough so as to align the secondary tool for operation in parallel or adjacent to operation along the guide wire, wherein the arm is formed with a radius of curvature selected to accommodate a curvature of a surface of the joint.

12. A method of intervening in subchondral bone to ameliorate one or more defects while preserving a glenohumeral or other joint of the shoulder using a hybrid subchondral implant-biologic member construct, the method comprising:
a) surgically opening access to a glenohumeral or other joint of the shoulder joint of the patient;
b) preparing a site of subchondral bone at the glenohumeral or other joint of the shoulder by removing a circular portion of the subchondral bone while preserving a central post of the subchondral bone in a substantially undisturbed native state;
c) preparing a hybrid subchondral implant-biologic member construct by:
1) forming a slit through a biologic member;
2) positioning a driver instrument through the slit;
3) engaging the driver instrument with a subchondral implant; and
4) securing the biologic member to the subchondral implant so that the hybrid subchondral implant-biologic member construct is formed; and
d) installing the hybrid subchondral implant-biologic member construct over a top of the central post in the glenohumeral or other joint of the shoulder,

13. The method of claim 12, wherein the installing comprises the hybrid subchondral implant-biologic construct comprises being introduced in a direction that is an antegrade approach relative to the joint.

14. The method of claim 12, wherein the installing comprises the hybrid subchondral implant-biologic construct comprises being introduced in a direction that is a retrograde approach relative to the joint.

15. The method of claim 12, wherein the installing comprises the hybrid subchondral implant-biologic construct comprises being introduced in a direction that is at an angle within 20-90 degrees with respect to a longitudinal axis of the joint or that is at another tangential approach relative to the joint.

16. The method of claim 11, wherein the hybrid subchondral implant-biologic construct includes a biologic graft of the same size, larger than, or asymmetric with respect to the subchondral implant.

17. The method of claim 16, wherein at least one of:
the biologic graft is attachable to a top and/or side(s) of the subchondral implant; or
the biologic graft includes sides and edges, the edges configured to be tucked down to cover one or more sides of the subchondral implant.

18. The method of claim 11, further comprising placing one or more than one additional hybrid subchondral implant-biologic construct into the one or more defects.

19. A method of intervening in subchondral bone to fuse a glenohumeral or other joint of the shoulder using a subchondral implant, the method comprising:
a) surgically opening access to a glenohumeral or other joint of the shoulder joint of the patient;
b) preparing a site of subchondral bone at the glenohumeral or other joint of the shoulder by removing damaged tissue in and/or between the subchondral bone of multiple bones along the joint so as to form a bore between the multiple bones along the joint;
c) preparing a subchondral implant; and
d) installing the subchondral implant construct in the bore so that the subchondral implant is engaging the subchondral bone of the multiple bones along the glenohumeral or other joint of the shoulder, wherein the subchondral implant engaging the subchondral bone of the multiple bones resists relative motion therebetween and thus facilitates fusion of the joint.

20. The method of claim 19, wherein the subchondral implant comprises a cylindrical body having a body outer diameter and a body inner diameter, wherein the subchondral implant further comprises a washer flange laterally extending from the cylindrical body away from the body outer diameter and to a washer outer diameter, wherein the washer engages surfaces of the multiple bones outward from the bore.
